(19) United States
(12) Patent Application Publication
(10) Pub. No.: US 2022/0218689 A1
(43) Pub. Date: Jul. 14, 2022

(54) TRANSDERMAL TROPANE COMPOSITIONS AND METHODS FOR USING THE SAME

(71) Applicant: Saniona A/S, Glostrup (DK)

(72) Inventors: Russell HAYNES, San Jose, CA (US); Jianye WEN, Palo Alto, CA (US); Bret BERNER, Seattle, WA (US)

(21) Appl. No.: 17/285,181

(22) PCT Filed: Oct. 24, 2019
(86) PCT No.: PCT/EP2019/079048

§ 371 Date: Apr. 14, 2021

Related U.S. Application Data
(60) Provisional application No. 62/750,043, filed on Oct. 24, 2018.

(51) Int. Cl. A61K 31/46; A61K 9/00; A61K 9/70; A61K 47/32

(52) U.S. Cl. CPC A61K 31/46; A61K 47/32; A61K 9/7061; A61K 9/0014

(57) ABSTRACT

Aspects of the invention include transdermal delivery devices for delivering a tropane active agent to a subject. Also provided are methods of using the subject transdermal delivery devices, as well as kits containing one or more of the transdermal delivery devices.

### CROSS-REFERENCE TO RELATED APPLICATIONS

This application is the National Stage of International Patent Application No. PCT/EP2019/079048, filed Oct. 24, 2019, which claims the benefit of U.S. Provisional Patent Application No. 62/750,043 filed Oct. 24, 2018, the disclosures of which are incorporated herein by reference in their entireties for any and all purposes.

### BACKGROUND

Cocaine is a pharmaceutical stimulant that is commonly known for its illicit and recreational use. Cocaine acts to block the reuptake of monoamines, dopamine, norepinephrine, and serotonin thus prolonging and magnifying the effects of these neurotransmitters in the central nervous system (Benowitz N L (1993) Pharmacol Toxicol 72, 3-12). Cocaine can be administered orally, intravenously, but is most often inhaled, such as by intranasal snorting.

Abuse of cocaine is an intractable social and medical problem that is resistant to remediation through pharmacotherapy. The abuse of cocaine and other stimulant drugs is a significant social and public health concern throughout the world (Crome, Drug Alcohol Dependence, 55:247, 1999). Cocaine dependence or addiction is generally associated with the physiological or psychological desire to use cocaine regularly and can lead to increased risk of stroke, myocardial infarction, lung ailments, infections and sudden cardiac arrest.

Cocaine toxicity is marked by both convulsions and cardiac dysfunction (e.g., myocardial infarction, cardiac arrhythmias, increased blood pressure, stroke, or dissecting aneurysm, and increased myocardial oxygen demand), due to effects on neurotransmitter systems and myocardial sodium channel blockade (Bauman J L and DiDomenico R J (2002) J Cardiovasc Pharmacol Ther 7, 195-202; Wilson L D and Shelat C (2003) J Toxicol Clin Toxicol 41, 777-788; Knuepfer M (2003) Pharmacol Ther 97, 181-222). Because of cocaine's ability to readily cross the blood brain barrier and its widespread effects on the central and peripheral nervous systems, overdose can result in sudden death (see Bauman J L and DiDomenico R J (2002) J Cardiovasc Pharmacol Ther 7, 195-202, for review).

### SUMMARY

Aspects of the invention include transdermal delivery devices for delivering a tropane active agent to a subject. The tropane active agent is a compound of Formula I (or a pharmaceutically acceptable salt thereof):

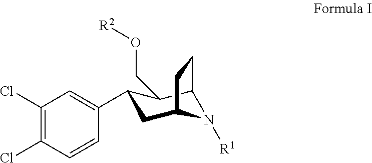

where

R¹=—H or —CH₃, and

R²=—CH₃ or —CH₂CH₃.

Accordingly, in some embodiments, the tropane active agent is (1 R,2 R,3 S,5 S)-3-(3,4-dichlorophenyl)-2-(methoxymethyl)-8-azabicyclo[3.2.1]octane (i.e., NS2359); (1 R,2 R,3 S,5 S)-3-(3,4-dichlorophenyl)-2-(ethoxymethyl)-8-methyl-8-azabicyclo[3.2.1]octane (i.e., Tesofensine or NS2330); or a pharmaceutically acceptable salt of these compounds. Also provided are methods of using the subject transdermal delivery devices, as well as kits containing the extended transdermal delivery device.

In some embodiments, the transdermal delivery device, e.g., patch, includes a backing layer and an active agent layer having a tropane active agent and a pressure sensitive adhesive. The tropane active agent may be present in the active agent layer in an amount that is 20% w/w or less, such as 10% w/w or less. In some instances, the pressure sensitive adhesive of the active agent layer includes an acrylic polymer, acrylate copolymer, acrylate-vinyl acetate copolymer or polyacrylonitrile or a mixture thereof. For example, the pressure sensitive adhesive may include an acrylate copolymer, such as an acrylate copolymer pendant having pendant hydroxyl functional groups, an acrylate copolymer having pendant carboxyl functional groups and an acrylate copolymer that lacks pendant functional groups or a mixture thereof. In one embodiment, the pressure sensitive adhesive of the active agent layer is an acrylate copolymer, such as an acrylate copolymer that lacks pendant functional groups. In some embodiments, the pressure sensitive includes a vinyl polymer, such as polyethylene, polypropylene, polyisobutylene, polybutene, polystyrene, polyvinyl chloride, polyvinyl acetate, polyvinyl alcohol, and organosilicones or a mixture thereof. The active agent layer may also include a hydrophilic polymer, such as polyvinylpyrrolidone, crosslinked polyvinylpyrrolidone, polyacrylic acid or crosslinked polyacrylic acid. The active agent layer may also include a 1-C₆₋₂₀ alkyl pyrrolidone, such as dodecyl pyrrolidone. In certain instances, the active agent layer may also include a compound such as lauryl lactate, propylene glycolmonolaurate, linolic acid, oleic acid, linolenic acid, stearic acid, isostearic acid, levulinic acid, palmitic acid, octanoic acid, decanoic acid, dodecanoic acid, tetradecanoic acid, hexadecanoic acid, stearic acid, N-lauroyl sarcosine, L-pyroglutamic acid, lauric acid, succinic acid, pyruvic acid, glutaric acid, sebacic acid, cyclopentane carboxylic acid and mixtures thereof. In one embodiment, the active agent layer further comprises a surfactant, such as selected from the group consisting of lauryl lactate, propylene glycolmonolaurate, N-lauroyl sarcosine, hexylene glycol, glyceryl monooleate and mixtures thereof. In one embodiment, the active agent layer further comprise a fatty acid, such as selected from the group consisting of linoleic acid, oleic acid, linolenic acid, stearic acid, isostearic acid, palmitic acid, octanoic acid, decanoic acid, dodecanoic acid, tetradecanoic acid, hexadecanoic acid, stearic acid, lauric acid, and mixtures thereof. In one embodiment, the active agent layer further comprises a compound selected from the group consisting of L-pyroglutamic acid, succinic acid, pyruvic acid, glutaric acid, sebacic acid, cyclopentane carboxylic acid, and mixtures thereof. In one embodiment, the compound is lauryl lactate. In one embodiment, the compound is propylene glycolmonolaurate. In one embodiment, the compound is hexylene glycol. In one embodiment, the compound is glyceryl monooleate. In one embodiment, the compound is linolic acid. In one embodiment, the compound is oleic acid. In one embodiment, the compound is linolenic acid. In one embodiment, the compound is stearic acid. In one embodiment, the compound is isostearic acid. In one embodiment, the compound is levulinic acid. In one embodiment, the compound is palmitic acid. In one embodiment, the compound is octanoic acid. In one embodiment, the compound is decanoic acid. In one embodiment, the compound is dodecanoic acid. In one embodiment, the compound is tetradecanoic acid. In one embodiment, the compound is hexadecanoic acid. In one embodiment, the compound is stearic acid. In one embodiment, the compound is N-lauroyl sarcosine. In one embodiment, the compound is L-pyroglutamic acid. In one embodiment, the compound is lauric acid. In one embodiment, the compound is succinic acid. In one embodiment, the compound is pyruvic acid. In one embodiment, the compound is glutaric acid. In one embodiment, the compound is sebacic acid. In one embodiment, the compound is cyclopentane carboxylic acid. In certain embodiments, the active agent layer includes a weak base. In some instances, the weak base is an amine, such as triethanolamine. In other instances, the weak base is a cationic acrylic copolymer, such as an aminated methacrylate copolymer.

In one embodiment, the active agent layer comprises the tropane active agent, an acrylate copolymer, and a hydrophilic polymer comprising polyvinylpyrrolidone. For example, the active agent layer comprises 2 to 10% wt/wt tropane active agent, 2 to 15% wt/wt hydrophilic polymer comprising polyvinylpyrrolidone, and 60 to 80% wt/wt acrylate copolymer. In certain embodiments, the active agent layer is in direct contact with the backing layer. In other embodiments, the transdermal delivery device includes an anchor layer positioned between the active agent layer and the backing layer. In some instances, the anchor layer is in direct contact with the backing layer. In other instances, the anchor layer is in direct contact with the backing layer and the active agent layer. The anchor layer may include a pressure sensitive adhesive, such as a polysiloxane, a polyisobutylene, a polyacrylate, a polyurethane, an ethylene-vinyl acetate copolymer, a low molecular weight polyether amide block polymer, a polyisobutene, a polystyrene-isoprene copolymer, a polystyrene-butadiene copolymer or a mixture thereof. The pressure sensitive adhesive of the anchor layer, in certain embodiments, is the same as the pressure sensitive adhesive of the active agent layer. In other embodiments, the pressure sensitive adhesive of the anchor layer is different from the pressure sensitive adhesive of the active agent layer.

Transdermal delivery devices may also include a conversion layer. The conversion layer is, in some instances, in contact with the active agent layer and when applied to a subject in contact with the skin of a subject. In some embodiments, the conversion layer includes a pressure sensitive adhesive such as an acrylic polymer, acrylate copolymer, acrylate-vinyl acetate copolymer or polyacrylonitrile or mixtures thereof. In certain instances, the pressure sensitive adhesive of the conversion layer is an acrylate copolymer, such as an acrylate copolymer pendant having pendant hydroxyl functional groups, an acrylate copolymer having pendant carboxyl functional groups and an acrylate copolymer that lacks pendant functional groups or a mixture thereof. In certain embodiments, the conversion layer includes a weak base. In some instances, the weak base is an amine, such as triethanolamine. In other instances, the weak base is a cationic acrylic copolymer, such as an aminated methacrylate copolymer. The pressure sensitive adhesive of the conversion layer is, in certain embodiments, the same as the pressure sensitive adhesive of the active agent layer. In other embodiments, the pressure sensitive adhesive of the conversion layer is different from the pressure sensitive adhesive of the active agent layer.

In other embodiments, the transdermal delivery devices are configured to provide a transdermal flux of the tropane active agent for an extended period of time which is within 90% or greater of peak transdermal flux when measured by an in-vitro protocol, such as for example protocols employing human cadaver skin with epidermal layers (stratum corneum and epidermis) in a Franz cell having donor and receptor sides clamped together and receptor solution containing phosphate buffer. For example, in these instances, the transdermal delivery device may be configured to provide a peak transdermal flux of the tropane active agent of 2.0 μg/cm²/hr or greater, such as 5 μg/cm²/hr or greater and including a peak flux of 7.0 μg/cm²/hr or greater. The transdermal delivery device may be configured to reach a peak flux of the tropane active agent within 8 hours or less, such as 5 hours or less and including 1 hour or less.

The transdermal delivery device may be configured to deliver a predetermined amount of the tropane active agent to a subject over an extended period of time, such as over 1 hour or longer, such as 3 hours or longer, such as 6 hours or longer, such as 12 hours or longer, such as 18 hours or longer, such as 1 day or longer, such as 2 days or longer, such as 3 days or longer, such as 5 days or longer and including 7 days or longer. In other embodiments, extended transdermal delivery devices of interest are configured to maintain a mean plasma concentration of the tropane active agent in the subject of from about 0.01 ng/mL to about 50 ng/mL.

Delivery of the tropane active agent by the subject transdermal delivery devices is, in some embodiments, a substantially first order rate. In other embodiments, delivery of the tropane active agent by the subject transdermal delivery devices is a substantially second order rate. In yet other embodiments, delivery of the tropane active agent by the subject transdermal delivery devices is characterized by a substantially second order rate for a first predetermined period of time and a substantially first order rate for a second predetermined period of time. For example, in this embodiment the first predetermined period of time may be 8 hours or less and the second predetermined period of time is 10 hours or more. In still other embodiments, delivery of the tropane active agent by the subject transdermal delivery devices is characterized by a substantially first order rate for a first predetermined period of time and a substantially zero order rate for a second predetermined period of time. For example, in this embodiment the first predetermined period of time may be 8 hours or less and the second predetermined period of time is 10 hours or more.

Aspects of the present disclosure also include methods for using the subject transdermal delivery devices. Methods according to certain embodiments include applying to a skin surface of a subject a transdermal delivery having a tropane active agent in a manner sufficient to deliver a therapeutically effective amount of the tropane active agent to the subject. In some embodiments, the transdermal delivery device is maintained in contact with the skin surface for 1 day or less, for example 12 hours or less. In other embodiments, the transdermal delivery device is maintained in contact with the skin surface for 3 days or more, such as 7 days or more.

In certain embodiments, methods include treating a subject for cocaine addiction with a tropane active agent by applying to a skin surface of a subject diagnosed as being addicted to cocaine a transdermal delivery device having a tropane active agent in a manner sufficient to deliver a therapeutically effective amount of the tropane active agent to the subject to treat the cocaine addiction. In some instances, the subject is diagnosed has having a psychological dependence on cocaine and methods include transdermally administering a tropane active agent to treat the psychological dependence on cocaine. In other instances, the subject is diagnosed has having a physiological dependence on cocaine and methods include transdermally administering an active agent to treat the physiological dependence on cocaine. In yet other instances, the subject is diagnosed has having both psychological and physiological dependence on cocaine and methods include transdermally administering a tropane active agent to treat both psychological and physiological dependence on cocaine. Methods may further include assessing the subject to determine that one or more of the psychological dependence on cocaine or physiological dependence on cocaine by the subject is reduced. In one embodiment, the transdermal delivery device described herein is for use in the treatment of cocaine addiction. Said cocaine addiction may include psychological and/or physiological dependence on cocaine. In one embodiment, when the tropane active agent of the transdermal delivery device is (1 R,2 R,3 S,5 S)-3-(3,4-dichlorophenyl)-2-(methoxymethyl)-8-azabicyclo[3.2.1]octane (i.e., NS2359), or a pharmaceutically acceptable salt thereof, the transdermal delivery device is for use in the treatment of cocaine addiction.

In other embodiments the methods include treating a subject suffering from obesity or an obesity related disorder as herein defined by applying to a skin surface of a subject suffering from obesity or an obesity related disorder a transdermal delivery device having a tropane active agent in a manner sufficient to deliver a therapeutically effective amount of the tropane active agent to the subject to treat obesity or an obesity related disorder. In one embodiment, the transdermal delivery device described herein is for use in the treatment of obesity or an obesity related disorder. In one embodiment, when the tropane active agent is (1 R,2 R,3 S,5 S)-3-(3,4-dichlorophenyl)-2-(ethoxymethyl)-8-methyl-8-azabicyclo[3.2.1]octane (i.e., Tesofensine or NS2330), or a pharmaceutically acceptable salt thereof, the transdermal delivery device is for use in the treatment of obesity or an obesity related disorder.

### DESCRIPTION OF DRAWINGS

FIG. 1 shows an example of the average flux of tropane compound (1 R,2 R,3 S,5 S)-3-(3,4-dichlorophenyl)-2-(methoxymethyl)-8-azabicyclo[3.2.1]octane as a function of transdermal delivery device application time according to certain embodiments as defined in Table 1.

FIG. 2 an example of a plot of the average cumulative amount of the tropane compound (1 R,2 R,3 S,5 S)-3-(3,4-dichlorophenyl)-2-(methoxymethyl)-8-azabicyclo[3.2.1]octane permeated as a function of time for transdermal delivery devices applied for a 72 hour dosage interval according to certain embodiments as defined in Table 1.

### DETAILED DESCRIPTION

Aspects of the invention include transdermal delivery devices for delivering a tropane active agent to a subject. Also provided are methods of using the subject transdermal delivery devices, as well as kits containing two or more of the subject transdermal delivery devices.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various embodiments of the invention, aspects of the transdermal delivery devices having a therapeutically effective amount of a tropane compound having serotonin, norepinephrine or dopamine reuptake inhibitor activity or a pharmaceutically acceptable salt thereof are reviewed first in greater detail, followed by a detailed description of embodiments of using the transdermal delivery devices, such as in the treatment of cocaine addiction and a review of kits that include the subject transdermal delivery devices.

Transdermal Delivery Devices Containing a Tropane Compound or a Pharmaceutically Acceptable Salt thereof

As described above, aspects of the present disclosure include transdermal delivery devices for delivering a therapeutically effective amount of a tropane active agent to a subject.

The term “transdermal” is used in its conventional sense to refer to the route of administration where an active agent (i.e., drug) is delivered across the skin (e.g., topical administration) for systemic distribution. As such, transdermal compositions containing a tropane active agent as described herein include compositions which are delivered to the subject through one or more of the subcutis, dermis and epidermis, including the stratum corneum, stratum germinativum, stratum spinosum and stratum basale. Accordingly, transdermal delivery devices having a composition containing a tropane active agent can be configured to be applied at any convenient location, such as for example, the arms, legs, buttocks, abdomen, back, neck, scrotum, face, behind the ear, etc. In describing methods of the present invention, the term “subject” is meant the person or organism to which the transdermal composition is applied and maintained in contact. As such, subjects of the invention may include but are not limited to mammals, e.g., humans and other primates, such as chimpanzees and other apes and monkey species; and pets like dogs and cats; and the like, where in certain embodiments the subject are humans.

In certain embodiments, transdermal delivery devices are configured for extended delivery of the tropane active agent to the subject. The term “extended delivery” is used herein to refer to a transdermal delivery device that includes a composition formulated to deliver the tropane active agent over an extended period of time, such as over the course of hours, days and including weeks, including 1 hour or longer, such as 2 hours or longer, such as 4 hours or longer, such as 8 hours or longer, such as 12 hours or longer, such as 24 hours or longer, such as 48 hours or longer, such as 72 hours or longer, such as 96 hours or longer, such as 120 hours or longer, such as 144 hours or longer and including 168 hours or longer. For the above ranges an upper limit period of time is, in some instances, 168 hours or shorter, such as 144 hours or shorter, such as 120 hours or shorter, such as 96 hours or shorter, such as 72 hours or shorter, such as 48 hours or shorter and including 24 hours or shorter. In certain embodiments, extended transdermal delivery ranges such as from 0.5 hours to 168 hours, such as from 1 hour to 144 hours, such as from 1.5 hours to 120 hours, such from 2 hours to 96 hours, such as from 2.5 hours to 72 hours, such as from 3 hours to 48 hours, such as from 3.5 hours to 24 hours, such as from 4 hours to 12 hours and including from 5 hours to 8 hours.

In some embodiments, sustained release transdermal administration of the tropane active agent includes multi-day delivery of a therapeutically effective amount of the tropane active agent that is applied to the skin of a subject. By multi-day delivery is meant that the transdermal composition is formulated to provide a therapeutically effective amount to a subject when the transdermal delivery device is applied to the skin of a subject for a period of time that is 1 day or longer, such as 2 days or longer, such as 4 days or longer, such as 7 days or longer, such as 14 days and including 30 days or longer. In certain embodiments, transdermal delivery devices provide a therapeutically effective amount of the tropane active agent to a subject for a period of 10 days or longer. For multi-day delivery, an upper limit period of time is, in some instances, 30 days or shorter, such as 28 days or shorter, such as 21 days or shorter, such as 14 days or shorter, such as 7 days or shorter and including 3 days or shorter. In certain embodiments, multi-day transdermal delivery ranges such as from 2 days to 30 days, such as from 3 days to 28 days, such as from 4 days to 21 days, such as from 5 days to 14 days and including from 6 days to 10 days.

The tropane active agent is a compound of Formula I (or a pharmaceutically acceptable salt thereof):

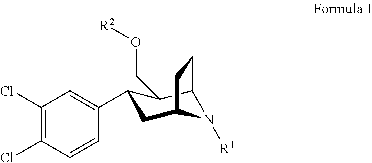

where

R¹=—H or —CH₃, and

R²=—CH₃ or —CH₂CH₃.

Accordingly, in some instances the tropane active agent is (1 R,2 R,3 S,5 S)-3-(3,4-dichlorophenyl)-2-(methoxymethyl)-8-azabicyclo[3.2.1]octane (i.e., NS2359) having the structure:

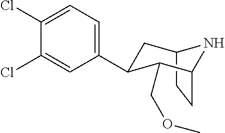 [SMILES: COCC1C2CCC(CC1c1ccc(Cl)c(Cl)c1)N2]

or a pharmaceutically acceptable salt thereof.

In some instances, the tropane active agent is (1 R,2 R,3 S,5 S)-3-(3,4-dichlorophenyl)-2-(ethoxymethyl)-8-methyl-8-azabicyclo[3.2.1]octane (i.e., Tesofensine or NS2330) having the structure:

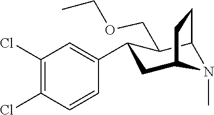

or a pharmaceutically acceptable salt thereof.

Transdermal delivery devices of interest may include the tropane active agent as a salt, such as a pharmaceutically acceptable salt, including but not limited to (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

Depending on the site of application and physiology of the subject and exposed surface area of the extended transdermal delivery device, the amount of the tropane active agent in transdermal compositions of interest may vary, in some instances, the amount of the tropane active agent ranging from 0.01 mg to 100 mg, such as from 0.02 mg to 90 mg, such as from 0.03 mg to 80 mg, such as from 0.04 mg to 70 mg, such as 0.05 mg to 60 mg, such as from 0.06 mg to 50 mg, such as from 0.07 mg to 40 mg, such as 0.08 mg to 30 mg, such as 0.09 mg to 20 mg, and including 0.01 mg to 10 mg. In some embodiments, the amount of the tropane active agent ranges from 10 mg to 50 mg, such as from 15 mg to 45 mg, such as from 20 mg to 40 mg and including from 25 mg to 35 mg. In certain embodiments, the amount of tropane active agent in the transdermal composition is from 1 mg to 2 mg, such as from 1.1 mg to 1.9 mg, such as from 1.2 mg to 1.8 mg, such as from 1.3 mg to 1.7 mg, such as from 1.4 mg to 1.6 and including 1.5 mg. In some embodiments, the amount of the tropane active agent in the transdermal composition is 20% w/w or less, such as 19% w/w or less, such as 18% w/w or less, such as 17% w/w or less, such as 16% w/w or less, such as 15% w/w or less, such as 14% w/w or less, such as 13% w/w or less, such as 12% w/w or less, such as 11% w/w or less, such as 10% w/w or less, such as 9% w/w or less, such as 8% w/w or less, such as 7% w/w or less, such as 6% w/w or less, such as 5% w/w or less, such as 4% w/w or less, such as 3% w/w or less, such as 2% w/w or less and including 1% w/w or less. In certain instances, the amount of the tropane active agent in the subject transdermal composition ranges from 1% w/w to 25% w/w, such as 2% w/w to 22.5% w/w, such as 3% w/w to 20% w/w, such as 4% w/w to 15% w/w and including 5% w/w to 10% w/w. In other embodiments, the amount of the tropane active agent in the subject transdermal compositions is 20% by mass or less of the total weight of the transdermal composition, such as 15% by mass or less, such as 10% by mass or less, such as 7% by mass or less, such as 5% by mass or less, such as 3% by mass or less and including 1% by mass or less of the total mass of the transdermal composition. In some instances, the amount of tropane active agent in the transdermal compositions ranges from 50 to 50,000 μg tropane active agent/cm², such as 500 to 20,000 μg tropane active agent/cm².

In embodiments of the present disclosure, transdermal compositions may also include one or more layers and a backing layer. In some embodiments, the subject transdermal delivery device includes an active agent layer and a backing layer. Depending on the dosage desired, the tropane active agent is present in the active agent layer in an amount that varies, in some instances, the amount of the tropane active agent in the active agent layer ranges from 0.01 mg to 100 mg, such as from 0.02 mg to 90 mg, such as from 0.03 mg to 80 mg, such as from 0.04 mg to 70 mg, such as 0.05 mg to 60 mg, such as from 0.06 mg to 50 mg, such as from 0.07 mg to 40 mg, such as 0.08 mg to 30 mg, such as 0.09 mg to 20 mg, and including 0.01 mg to 10 mg. In some embodiments, the amount of the tropane active agent in the active agent layer ranges from 10 mg to 50 mg, such as from 15 mg to 45 mg, such as from 20 mg to 40 mg and including from 25 mg to 35 mg. In certain embodiments, the amount of tropane active agent in the active agent layer is from 1 mg to 2 mg, such as from 1.1 mg to 1.9 mg, such as from 1.2 mg to 1.8 mg, such as from 1.3 mg to 1.7 mg, such as from 1.4 mg to 1.6 and including 1.5 mg. In some embodiments, the amount of the tropane active agent in the active agent layer is 20% w/w or less, such as 19% w/w or less, such as 18% w/w or less, such as 17% w/w or less, such as 16% w/w or less, such as 15% w/w or less, such as 14% w/w or less, such as 13% w/w or less, such as 12% w/w or less, such as 11% w/w or less, such as 10% w/w or less, such as 9% w/w or less, such as 8% w/w or less, such as 7% w/w or less, such as 6% w/w or less, such as 5% w/w or less, such as 4% w/w or less, such as 3% w/w or less, such as 2% w/w or less and including 1% w/w or less. In certain instances, the amount of the tropane active agent in the active agent layer ranges from 1% w/w to 25% w/w, such as 2% w/w to 22.5% w/w, such as 3% w/w to 20% w/w, such as 4% w/w to 15% w/w and including 5% w/w to 10% w/w. In other embodiments, the amount of the tropane active agent in the active agent layer is 20% by mass or less of the total weight of the transdermal composition, such as 15% by mass or less, such as 10% by mass or less, such as 7% by mass or less, such as 5% by mass or less, such as 3% by mass or less and including 1% by mass or less of the total mass of the transdermal composition.

In embodiments, the active agent layer includes a therapeutically effective amount of a tropane active agent as described above and a pressure sensitive adhesive. Pressure sensitive adhesives of interest may include but are not limited to poly-isobutene adhesives, poly-isobutylene adhesives, poly-isobutene/polyisobutylene adhesive mixtures, carboxylated polymers, acrylic or acrylate copolymers, such as carboxylated acrylate copolymers, acrylate-vinyl acetate copolymer or polyacrylonitrile polymers or mixtures thereof. Acrylate copolymers of interest include copolymers of various monomers, such as “soft” monomers, “hard” monomers or “functional” monomers. The acrylate copolymers can be composed of a copolymer including bipolymer (i.e., made with two monomers), a terpolymer (i.e., made with three monomers), or a tetrapolymer (i.e., made with four monomers), or copolymers having greater numbers of monomers. The acrylate copolymers may be crosslinked or non-crosslinked. The polymers can be cross-linked by known methods to provide the desired polymers. The monomers from of the acrylate copolymers may include at least two or more exemplary components selected from the group including acrylic acids, alkyl acrylates, methacrylates, copolymerizable secondary monomers or monomers with functional groups. Monomers (“soft” and “hard” monomers) may be methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. Additional examples of acrylic adhesive monomers are described in Satas, “Acrylic Adhesives,” Handbook of Pressure-Sensitive Adhesive Technology, 2nd ed., pp. 396-456 (D. Satas, ed.), Van Nostrand Reinhold, New York (1989), the disclosure of which is herein incorporated by reference. In some embodiments, the pressure sensitive adhesive is an acrylate-vinyl acetate copolymer. In one embodiment, the pressure sensitive adhesive of the active agent layer is an acrylate copolymer, such as an acrylate copolymer that lacks pendant functional groups. In some embodiments, the pressure sensitive adhesive may include a composition that is, or is substantially the same as, the composition of Duro-Take 87-9301, Duro-Take 87-900A, Duro-Take 87-200A, Duro-Tak®87-2353, Duro-Take87-2100, Duro-Take87-2051, Duro-Tak®87-2052, Duro-Take87-2194, Duro-Tak®87-2677, Duro-Take87-201A, Duro-Tak®87-2979, Duro-Take87-2510, Duro-Take87-2516, Duro-Tak®87-387, Duro-Tak®87-4287, Duro-Tak®87-2287,and Duro-Tak®87-2074 and combinations thereof. The term “substantially the same” as used herein refers to a composition that is an acrylate-vinyl acetate copolymer in an organic solvent solution. In certain embodiments, the acrylic pressure-sensitive adhesive is Duro-Tak® 87-2054. In one embodiment, the acrylic pressure-sensitive adhesive is Duro-Tak® 87-9301. In one embodiment, the acrylic pressure-sensitive adhesive is Duro-Tak® 87-900A.

In certain embodiments, the pressure sensitive adhesive is an acrylate adhesive that is a non-functionalized acrylate, hydroxyl-functionalized acrylate or an acid functionalized acrylate. For example, the acrylate adhesive may be an acrylic adhesive having one or more pendant —OH functional groups. Where the acrylic adhesive has one or more pendant —OH functional groups, in some instances, the pressure sensitive adhesive may be a composition that is, or is substantially the same as, the composition of Duro-Take 87-4287, Duro-Take 87-2287, Duro-Take 87-2510 and Duro-Take 87-2516 and combinations thereof. The acrylate adhesive may alternatively be an acrylic adhesive having one or more pendant —COOH functional groups. Where the acrylic adhesive has one or more pendant —COOH functional groups, in some instances, the pressure sensitive adhesive may be a composition that is or is substantially the same as, the composition of Duro-Take 87-387, Duro-Take 87-2979 and Duro-Take 87-2353 and combinations thereof. Still further, the acrylate adhesive may be a non-functionalized acrylic adhesive. In these embodiments, the acrylate adhesive may be an acrylate copolymer that lacks pendant functional groups. Where the acrylic adhesive is non-functionalized, in some instances the pressure sensitive adhesive may be a composition that is or is substantially the same as, the composition of Duro-Take 87-9301.

In one embodiment, the pressure-sensitive adhesive is an acrylate copolymer that is non-functionalized. In one embodiment, the pressure-sensitive adhesive is an acrylate copolymer which does not contain vinyl acetate. In one embodiment, the pressure-sensitive adhesive is an acrylate copolymer with a viscosity of no more than 15000 mPa·s, such as no more than 10000 mPa·s, such as no more than 7500 mPa·s, such as no more than 5000 mPa·s, such as no more than 2000 mPa·s. In one embodiment, the pressure-sensitive adhesive is an acrylate copolymer with a viscosity of at least 1000 mPa·s. In one embodiment, the pressure-sensitive adhesive is an acrylate copolymer with a viscosity in the range of 1000 to 10000 mPa·s, for example in the range of 1000 to 5000 mPa·s or in the range of 5000 to 10000 mPa·s. In one embodiment, the pressure-sensitive adhesive is an acrylate copolymer that is non-functionalized and has a viscosity of no more than 5000 mPa·s, In one embodiment, the pressure-sensitive adhesive is an acrylate copolymer that is non-functionalized and has a viscosity in the range of 8000 to 12000 mPa·s,

The amount of pressure sensitive adhesive in transdermal compositions of interest may vary, the amount of pressure sensitive adhesive ranging from 0.1 mg to 2000 mg, such as 0.5 mg to 1500 mg, such as 1 to 1000 mg, such as 10 to 750 mg, and including 10 mg to 500 mg. As such, the amount of pressure sensitive adhesive in the transdermal composition ranges from 1% to 99% w/w, such as 5% to 95% w/w, such as 10% to 95%, such as 15% to 90% w/w and including 20% to 85% w/w. In other embodiments, the amount of pressure sensitive adhesive in the subject transdermal compositions is 70% by weight or greater of the total weight of the transdermal composition, such as 75% by weight or greater, such as 80% by weight or greater, such as 85% by weight or greater, such as 90% by weight or greater, such as 95% by weight or greater and including 97% by weight or greater of the total weight of the transdermal composition.

The weight ratio of the tropane active agent to pressure sensitive adhesive in the subject compositions may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; 1:10 and 1:25; 1:25 and 1:50; 1:50 and 1:75; and 1:75 and 1:99 or a range thereof. For example, the weight ratio of the tropane active agent to pressure sensitive adhesive in compositions of interest may range between 1:1 and 1:5; 1:5 and 1:10; 1:10 and 1:15; 1:15 and 1:25; 1:25 and 1:50; 1:50 and 1:75 or 1:75 and 1:99. Alternatively, the weight ratio of pressure sensitive adhesive to the tropane active agent in the subject compositions ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; 10:1 and 25:1; 25:1 and 50:1; 50:1 and 75:1; and 75:1 and 99:1 ora range thereof. For example, the ratio of pressure sensitive adhesive to the tropane active agent in compositions of interest may range between 1:1 and 5:1; 5:1 and 10:1; 10:1 and 15:1; 15:1 and 25:1; 25:1 and 50:1; 50:1 and 75:1; or 75:1 and 99:1.

In some embodiments, the transdermal delivery device is configured with a single layer transdermal tropane composition. By “single layer” is meant that the transdermal delivery device includes only a single layer of the tropane composition having a tropane active agent incorporated into a pressure sensitive adhesive in contact with the backing layer and does not include any additional distinct layers. In these embodiments, the transdermal delivery device includes an active agent layer having a pressure sensitive adhesive and a tropane active agent in contact with the backing layer. Where the active agent layer further includes one or more of an enhancer (e.g., solubility, permeation, percutaneous absorption), hydrophilic polymer (e.g., PVP, PVP-K90, PVP-CLM), weak base (e.g., cationic aminated copolymer), each additional component is incorporated into the active agent layer and a single active agent layer is in contact with the backing layer. Single layer transdermal delivery devices of the present disclosure do not include separate layers for the tropane compound active agent and pressure sensitive adhesive or do not further include a separate reservoir for the tropane compound or pharmaceutically acceptable salt thereof separate from the pressure sensitive adhesive. Depending on the length of the dosage interval and the desired target dosage, the thickness of single layer matrices of interest may vary, in some instances ranging in thickness from 10-200 micron, such as from 20-175 micron, such as 25-150 micron, such as 35-140 micron, such as 40-125 micron mm and including 50-100 micron.

In certain embodiments, the subject transdermal delivery devices further include one or more additional layers. In these embodiments, the transdermal delivery device includes an active agent layer having a tropane active agent in a pressure sensitive adhesive and one or more distinct layers, such as 2 or more layers, such as 3 or more layers, such as 4 or more layers, such as 5 or more layers and including 6 or more layers. As described in greater detail below, in certain instances the subject transdermal delivery devices may include one or more active agent layers having the tropane active agent and a pressure sensitive adhesive and one or more anchor layers. In other instances, the subject transdermal delivery devices may include one or more active agent layers having the tropane active agent and a pressure sensitive adhesive and one or more conversion layers. In yet other instances, the subject transdermal delivery devices may include one or more active agent layers having the tropane active agent and a pressure sensitive adhesive and one or more anchor layers as well as one or more conversion layers.

In some instances, transdermal delivery devices further include an anchor layer. For example, the anchor layer may be a layer that is positioned between the active agent layer and the backing layer. In these embodiments, the anchor layer is in contact with the backing layer. In embodiments, the anchor layer may include a pressure sensitive adhesive, such as those described above for the active agent layer. For example, the pressure sensitive adhesive of the anchor layer may be composed of polysiloxanes, polyisobutylenes, polyacrylates, polyurethanes, ethylene-vinyl acetate copolymers, low molecular weight polyether amide block polymers, polyisobutene, polystyrene-isoprene copolymers, polystyrene-butadiene copolymers or mixtures thereof. In some instances, the pressure sensitive adhesive of the anchor layer is an acrylate copolymer. The acrylate copolymers may be crosslinked or non-crosslinked. The monomers from of the acrylate copolymers may include at least two or more exemplary components selected from the group including acrylic acids, alkyl acrylates, methacrylates, copolymerizable secondary monomers or monomers with functional groups. Monomers (“soft” and “hard” monomers) may be methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. In certain embodiments, the pressure sensitive adhesive of the anchor layer is an acrylate adhesive that is a non-functionalized acrylate, hydroxyl-functionalized acrylate or an acid functionalized acrylate. For example, the acrylate adhesive may be an acrylic adhesive having one or more pendant —OH functional groups. The acrylate adhesive may alternatively be an acrylic adhesive having one or more pendant —COOH functional groups. Still further, the acrylate adhesive may be a non-functionalized acrylic adhesive. In these embodiments, the acrylate adhesive may be an acrylate copolymer that lacks pendant functional groups.

The thickness of the anchor layer depends on the size of the subject transdermal delivery device (as described in greater detail below) and the thickness of the active agent layer. In some instances, the anchor layer ranges in thickness from 10-200 micron, such as from 20-175 micron, such as 25-150 micron, such as 35-140 micron, such as 40-125 micron mm and including 50-100 micron. In embodiments, the ratio of the thickness of the anchor layer and the active agent layer may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; and 1:10 and 1:25 or a range thereof. For example, the ratio of the thickness of the anchor layer and the active agent layer in transdermal delivery devices of interest may range between 1:1 and 1:5; 1:5 and 1:10; 1:10 and 1:15; or 1:15 and 1:25. Alternatively, the ratio of the thickness of the active agent layer and the anchor layer ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; and 10:1 and 25:1 or a range thereof. For example, the ratio of the thickness of the active agent layer and the anchor layer in transdermal delivery devices of interest may range between 1:1 and 5:1; 5:1 and 10:1; 10:1 and 15:1; or 15:1 and 25:1.

In certain embodiments, the subject transdermal delivery devices further include a conversion layer. In these embodiments, the conversion layer may be in contact with the active agent layer and is configured to be positioned onto the surface of the skin of the subject. In some instances, the conversion layer (i.e., converting layer, converting matrix or active agent conversion layer) is a layer that serves to convert the a salt of the tropane compound in the active agent layer to free base form upon application of the composition to skin. The conversion layer is characterized, at least during storage and prior to skin contact or use, as having substantially less active agent (e.g., 5% by weight or less, such as 2.5% by weight or less, including 1% by weight or less) than the active agent layer, where in some instances the conversion layer includes substantially no active agent (e.g., 0.9% by weight or less, such as 0.5% or less, including no detectable active agent). The conversion layer includes a converting agent, which may be any agent that can mediate the conversion (either alone or in combination with one or more other components) the active agent from salt form to free base form. Converting agents of interest that may be present in the converting layer include weak bases. By weak base is meant a base having a base dissociation constant (Kb) of 10 or less, such as 9 or less. Any convenient weak base may be employed, such as polymeric weak bases, e.g., cationic acrylic copolymers, inorganic bases, e.g., calcium hydroxide; etc. Cationic acrylic copolymers of interest are polymers of two or more different monomeric residues, where at least one of the residues is an acrylic residue, e.g., an acrylate or a methacrylate, and at least one of the residues includes a cationic pendant group, e.g., an amino pendant group, where these features may be included in the same or different monomeric residues making up the copolymer. Where desired, the cationic acrylic copolymer may be an aminated methacrylate copolymer. The aminated methacrylate copolymer may be a copolymer of diethylaminoethyl methacrylate, butyl methacrylate and methyl methacrylate. Of interest are aminated methacrylate copolymers, such as aminated methacrylate copolymers that are substantially the same as Eudragit® E100 aminated methacrylate copolymer. As used herein, the term “substantially the same” means that the aminated methacrylate copolymer has the same functional impact on the composition as Eudragit® E100 aminated methacrylate copolymer. In some instances, the aminated methacrylate copolymer is Eudragit® E100 aminated methacrylate copolymer. If present, the amount of cationic acrylic copolymer may be present in an amount ranging from 1 to 15, such as 2 to 10 and including 4 to 8% by weight of the converting layer.

In addition to the converting agent, e.g., weak base, the conversion layer further includes a pressure sensitive adhesive. In some embodiments, the conversion layer includes a pressure sensitive adhesive, such as those described above for the active agent layer. For example, the pressure sensitive adhesive of the anchor layer may be composed of polysiloxanes, polyisobutylenes, polyacrylates, polyurethanes, ethylene-vinyl acetate copolymers, low molecular weight polyether amide block polymers, polyisobutene, polystyrene-isoprene copolymers, polystyrene-butadiene copolymers or mixtures thereof. In some instances, the pressure sensitive adhesive of the anchor layer is an acrylate copolymer. The acrylate copolymers may be crosslinked or non-crosslinked. The monomers from of the acrylate copolymers may include at least two or more exemplary components selected from the group including acrylic acids, alkyl acrylates, methacrylates, copolymerizable secondary monomers or monomers with functional groups. Monomers (“soft” and “hard” monomers) may be methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. In certain embodiments, the pressure sensitive adhesive of the anchor layer is an acrylate adhesive that is a non-functionalized acrylate, hydroxyl-functionalized acrylate or an acid functionalized acrylate. For example, the acrylate adhesive may be an acrylic adhesive having one or more pendant —OH functional groups. The acrylate adhesive may alternatively be an acrylic adhesive having one or more pendant —COOH functional groups. Still further, the acrylate adhesive may be a non-functionalized acrylic adhesive. In these embodiments, the acrylate adhesive may be an acrylate copolymer that lacks pendant functional groups.

In some embodiments, of interest are acrylate copolymers that include polar functional monomeric residues. Of specific interest are monomeric residues that provide for —COOH functional groups. Useful carboxylic acid monomers to provide the —COOH functional group may contain from about 3 to about 6 carbon atoms and include, among others, acrylic acid, methacrylic acid, itaconic acid, and the like. Acrylic acid, methacrylic acid and mixtures thereof are employed in certain embodiments. The functional monomer(s) are present in certain embodiments of the copolymers in an amount of 2 wt % or more, such as between 3-10 wt %.

In some instances, the pressure sensitive adhesive is present in the conversion layer in an amount of from 50% w/w to 95% w/w, such as 60% w/w to 90% w/w and including 65% w/w to 85% w/w of the conversion layer.

In addition to the weak base, the conversion layer may also include a carboxylated component. By carboxylated component is meant a component of the layer, e.g., the matrix or an additional compound in the matrix, that has a carboxyl moiety. As such, in some instances the carboxylated component is a polymeric component, such as a matrix component, e.g., a carboxylated polymer of a pressure sensitive adhesive, such as described above. Additionally or alternatively, the carboxylated component may be some other compound present in the conversion layer, such as small molecule, e.g., an organic acid, e.g., ascorbic acid.

The thickness of the conversion layer depends on the size of the subject transdermal delivery device (as described in greater detail below) and the thickness of the active agent layer. In some instances, the conversion layer ranges in thickness from 10-200 micron, such as from 20-175 micron, such as 25-150 micron, such as 35-140 micron, such as 40-125 micron mm and including 50-100 micron. In embodiments, the ratio of the thickness of the conversion layer and the active agent layer may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; and 1:10 and 1:25 or a range thereof. For example, the ratio of the thickness of the conversion layer and the active agent layer in transdermal delivery devices of interest may range between 1:1 and 1:5; 1:5 and 1:10; 1:10 and 1:15; or 1:15 and 1:25. Alternatively, the ratio of the thickness of the active agent layer and the conversion layer ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; and 10:1 and 25:1 ora range thereof.

For example, the ratio of the thickness of the active agent layer and the conversion layer in transdermal delivery devices of interest may range between 1:1 and 5:1; 5:1 and 10:1; 10:1 and 15:1; or 15:1 and 25:1.

In some embodiments, one or more of the active agent layer, conversion layer and anchor layer may further include one or more hydrophilic polymers, such as a crosslinked hydrophilic polymer. For example, the hydrophilic polymer may be an amine-containing hydrophilic polymer. Amine-containing polymers may include, but are not limited to, polyethyleneimine, amine-terminated polyethylene oxide, amine-terminated polyethylene/polypropylene oxide, polymers of dimethyl amino ethyl methacrylate, and copolymers of dimethyl amino ethyl methacrylate and vinyl pyrrolidone. In some embodiments, the hydrophilic polymer is polyvinylpyrrolidone. In certain embodiments, the hydrophilic polymer is a crosslinked polymer, such as crosslinked polyvinylpyrrolidone, such as for example PVP-CLM. The hydrophilic polymer may be PVP K17, PVP K30 or PVP K90 that inhibit crystallization of the tropane compound, have hygroscopic properties that improve the duration of wear, and improve the physical properties, e.g., cold flow, tack, cohesive strength, of the pressure sensitive adhesive in the respective layer (e.g., active agent layer).

The amount of hydrophilic polymer (crosslinked hydrophilic polymer) may vary, ranging from 0.1 mg to 500 mg, such as 0.5 mg to 400 mg, such as 1 to 300 mg, such as 10 to 200 mg, and including 10 mg to 100 mg. As such, the amount of hydrophilic polymer in the layer (e.g., active agent layer, conversion layer, anchor layer, etc.) ranges from 2% to 30% w/w, such as 4% to 30% w/w, such as 5% to 25%, such as 6% to 22.5% w/w and including 10% to 20% w/w. In other embodiments, the amount of hydrophilic polymer is 8% by weight or greater of the total weight of the layer, such as 10% by weight or greater, such as 12% by weight or greater, such as 15% by weight or greater, such as 20% by weight or greater, such as 25% by weight or greater and including 30% by weight hydrophilic polymer or greater of the total weight of the layer (e.g., active agent layer, conversion layer, anchor layer, etc.).

In certain embodiments, one or more of the active agent layer, conversion layer and anchor layer may further include an enhancer. The enhancer may be a solubility enhancer, a permeation enhancer, a percutaneous absorption enhancer or other type of enhancer that improves the physical properties of the respective layer in the transdermal device, such as preventing crystallization of the tropane compound in the active agent layer or improves the stability or rheological properties of the conversion or anchor layers. Where present, the enhancer is incorporated into one or more layers of the transdermal delivery device, the amount of enhancer in the respective layer (e.g., active agent layer, anchor layer, conversion layer, etc.) independently ranges from 0.01% to 20% (w/w), such as from 0.05% to 15% (w/w), such as from 0.1% to 10% (w/w), such as from 0.5% to 8% (w/w) and including from 1% to 5% (w/w).

Example enhancers include, but are not limited to acids including linolic acid, oleic acid, linolenic acid, stearic acid, isostearic acid, levulinic acid, palmitic acid, octanoic acid, decanoic acid, dodecanoic acid, tetradecanoic acid, hexadecanoic acid, octadecanoic acid (i.e., stearic acid), N-lauroyl sarcosine, L-pyroglutamic acid, lauric acid, succinic acid, pyruvic acid, glutaric acid, sebacic acid, cyclopentane carboxylic acid; acylated amino acids. Other solubility enhancers of interest may include, but is not limited to aliphatic alcohols, such as saturated or unsaturated higher alcohols having 12 to 22 carbon atoms (e.g., oleyl alcohol or lauryl alcohol); fatty acid esters, such as isopropyl myristate, diisopropyl adipate, lauryl lactate, propyl laurate, ethyl oleate and isopropyl palmitate; alcohol amines, such as triethanolamine, triethanolamine hydrochloride, and diisopropanolamine; polyhydric alcohol alkyl ethers, such as alkyl ethers of polyhydric alcohols such as glycerol, ethylene glycol, propylene glycol, 1,3-butylene glycol, diglycerol, polyglycerol, diethylene glycol, polyethylene glycol, dipropylene glycol, polypropylene glycol, polypropylene glycolmonolaurate, sorbitan, sorbitol, isosorbide, methyl glucoside, oligosaccharides, and reducing oligosaccharides, where the number of carbon atoms of the alkyl group moiety in the polyhydric alcohol alkyl ethers is preferably 6 to 20; polyoxyethylene alkyl ethers, such as polyoxyethylene alkyl ethers in which the number of carbon atoms of the alkyl group moiety is 6 to 20, and the number of repeating units (e.g. —O—CH₂CH₂—) of the polyoxyethylene chain is 1 to 9, such as but not limited to polyoxyethylene lauryl ether, polyoxyethylene cetyl ether, polyoxyethylene stearyl ether, and polyoxyethylene oleyl ether; glycerides (i.e., fatty acid esters of glycerol), such as glycerol esters of fatty acids having 6 to 18 carbon atoms, where the glycerides may be monoglycerides (i.e., a glycerol molecule covalently bonded to one fatty acid chain through an ester linkage), diglycerides (i.e., a glycerol molecule covalently bonded to two fatty acid chains through ester linkages), triglycerides (i.e., a glycerol molecule covalently bonded to three fatty acid chains through ester linkages), or combinations thereof, where the fatty acid components forming the glycerides include octanoic acid, decanoic acid, dodecanoic acid, tetradecanoic acid, hexadecanoic acid, octadecanoic acid (i.e., stearic acid) and oleic acid; middle-chain fatty acid esters of polyhydric alcohols; lactic acid alkyl esters; dibasic acid alkyl esters; acylated amino acids; pyrrolidone;

pyrrolidone derivatives and combinations thereof. Additional types of enhancers may include lactic acid, tartaric acid, 1,2,6-hexanetriol, benzyl alcohol, lanoline, potassium hydroxide (KOH), tris(hydroxymethyl)aminomethane, glyceryl monooleate (GMO), sorbitan monolaurate (SML), sorbitan monooleate (SMO), laureth-4 (LTH), and combinations thereof. In certain embodiments, the enhancer is levulinic acid, lauryl lactate or propylene glycolmonolaurate. In one embodiment, the enhancer is a hydrophilic polymer, such as polyvinylpyrrolidone, crosslinked polyvinylpyrrolidone, polyacrylic acid or crosslinked polyacrylic acid. The enhancer may also include a 1-C₆₋₂₀alkyl pyrrolidone, such as dodecyl pyrrolidone. In one embodiment, the enhancer is a surfactant, such as selected from the group consisting of lauryl lactate, propylene glycolmonolaurate, N-lauroyl sarcosine, hexylene glycol, glyceryl monooleate and mixtures thereof. In one embodiment, the enhancer is a fatty acid, such as selected from the group consisting of linoleic acid, oleic acid, linolenic acid, stearic acid, isostearic acid, palmitic acid, octanoic acid, decanoic acid, dodecanoic acid, tetradecanoic acid, hexadecanoic acid, stearic acid, lauric acid, and mixtures thereof. In one embodiment, the enhancer is a compound selected from the group consisting of L-pyroglutamic acid, succinic acid, pyruvic acid, glutaric acid, sebacic acid, cyclopentane carboxylic acid, and mixtures thereof. In one embodiment, the enhancer is selected from the group consisting of PVP, lauryl lactate, glyceryl monooleate, dodecyl pyrrolidone, hexylene glycol and mixtures thereof. In one embodiment, the enhancer is lauryl lactate. In one embodiment, the enhancer is propylene glycolmonolaurate. In one embodiment, the enhancer is hexylene glycol. In one embodiment, the enhancer is glyceryl monooleate. In one embodiment, the enhancer is linolic acid. In one embodiment, the enhancer is oleic acid. In one embodiment, the enhancer is linolenic acid. In one embodiment, the enhancer is stearic acid. In one embodiment, the enhancer is isostearic acid. In one embodiment, the enhancer is levulinic acid. In one embodiment, the enhancer is palmitic acid. In one embodiment, the enhancer is octanoic acid. In one embodiment, the enhancer is decanoic acid. In one embodiment, the enhancer is dodecanoic acid. In one embodiment, the enhancer is tetradecanoic acid. In one embodiment, the enhancer is hexadecanoic acid. In one embodiment, the enhancer is stearic acid. In one embodiment, the enhancer is N-lauroyl sarcosine. In one embodiment, the enhancer is L-pyroglutamic acid. In one embodiment, the enhancer is lauric acid. In one embodiment, the enhancer is succinic acid. In one embodiment, the enhancer is pyruvic acid. In one embodiment, the enhancer is glutaric acid. In one embodiment, the enhancer is sebacic acid. In one embodiment, the enhancer is cyclopentane carboxylic acid. In certain embodiments, the active agent layer includes a weak base. In some instances, the weak base is an amine, such as triethanolamine. In other instances, the weak base is a cationic acrylic copolymer, such as an aminated methacrylate copolymer.

In some embodiments, one or more of the active agent layer, conversion layer and anchor layer may further include one or more methacrylic acid polymers. By “methacrylic acid polymer” is meant the class of polymeric compounds described by the formula:

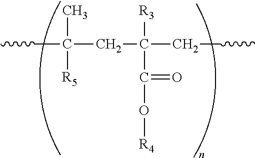

wherein R₄ is —H or a C1-C12 alkyl; R₅ is an anionic moiety, such as a carboxylic acid (i.e., —COOH) and R₃ is —H or a C1-C12 alkyl. The size of the methacrylic acid copolymer may vary, where n may be 50 or greater, such as 75 or greater, such as 100 or greater, such as 200 or greater, such as 350 or greater, such as 500 or greater and including 750 or greater. As such, the molecular weight of the subject methacrylic acid copolymer may be 5 kDa or greater, such as 10 kDa or greater, such as 25 kDa or greater, such as 50 kDa or greater, such as 60 kDa or greater, such as 70 kDa or greater, such as 100 kDa or greater, such as 135 kDA or greater and including 150 kDa or greater.

In certain embodiments, R₄ and R₃ is alkyl and R₅ is an anionic moiety, such as a carboxylic acid. In these embodiments, R₄ and R₃ may be methyl, ethyl, propyl, butyl, pentyl, isobutyl, isopropyl, tert-butyl, among other straight chain or branched alkyls and R₅ may be a carboxylic acid. For example, in certain instances, R₄ is ethyl, R₅ is carboxylic acid and R₃ is methyl.

In some embodiments, the methacrylic acid polymer is substantially the same as a Eudragit® methacrylic acid copolymer. The term “Eudragit® methacrylic acid copolymer” is used in its conventional sense to refer to copolymers derived from esters of acrylic and methacrylic acid. In embodiments of the invention, Eudragit® polymers may be methacrylic acid copolymers (e.g., functional group being carboxylic acid). In certain embodiments, the Eudragit® polymer is a methacrylic acid Eudragit® polymer, such as Eudragit® L100 or Eudragit® L100-55.

In some instances, one or more of the active agent layer, conversion layer and anchor layer may further include a weak base, such as a cationic acrylic copolymer. Cationic acrylic copolymers of interest are polymers of two or more different monomeric residues, where at least one of the residues is an acrylic residue, e.g., an acrylate or a methacrylate, and at least one of the residues includes a cationic pendant group, e.g., an amino pendant group, where these features may be includes in the same or different monomeric residues making up the copolymer. Where desired, the cationic acrylic copolymer may be aminated methacrylate copolymer. The aminated methacrylate copolymer may be a copolymer of diethylaminoethyl methacrylate, butyl methacrylate and methyl methacrylate, e.g., present in a 2:1:1 ratio. In some instances, the aminated methacrylate copolymer may be described by the formula:

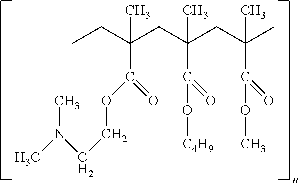

In some embodiments, the aminated methacrylate copolymer may be described by the formula:

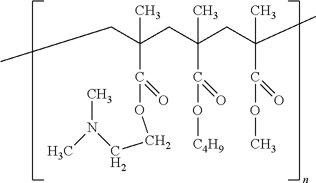

The size of the methacrylic acid copolymer may vary, where n may be 50 or greater, such as 75 or greater, such as 100 or greater, such as 200 or greater, such as 350 or greater, such as 500 or greater and including 750 or greater.

In some instances, the average molecular weight of the aminated methacrylate copolymer ranges from 25,000 to 75,000 daltons, such as 30,000 to 60,000 daltons, including 45,000 to 55,000 daltons.

Of interest are aminated methacrylate copolymers that are substantially the same as Eudragit® E100, Eudragit® E PO and/or Eudragit® E 12,5 aminated methacrylate copolymers. As used herein, the term substantially the same is meant that the aminated methacrylate copolymer has the same functional impact on the composition as Eudragit® E100, Eudragit® E PO and/or Eudragit® E 12,5 aminated methacrylate copolymers. In some instances, the aminated methacrylate copolymer is Eudragit® E100, Eudragit® E PO and/or Eudragit® E 12,5 aminated methacrylate copolymers.

In some embodiments, transdermal compositions of interest having the tropane compound or pharmaceutically acceptable salt thereof includes little to no water. In these embodiments, the amount of water in the subject transdermal compositions is 3% w/w or less, such as 2% w/w or less, such as 1% w/w or less, such as 0.5% w/w or less, such as 0.1% w/w or less, such as 0.01% w/w or less, such as 0.001% w/w or less, such as 0.0001% w/w or less, such as 0.00001% w/w or less and including 0.000001% w/w or less. In certain embodiments, the transdermal composition contains no water (i.e., 0% w/w water)

In certain embodiments, transdermal compositions of interest are aqueous compositions that include water. The amount of water in transdermal compositions of interest may vary, the amount of water ranging from 0.1 mg to 500 mg, such as 0.5 mg to 400 mg, such as 1 to 300 mg, such as 10 to 200 mg, and including 10 mg to 100 mg. As such, the amount of water in the transdermal composition ranges from 0.01% to 2% w/w, such as 0.025% to 1.5% w/w, such as 0.05% to 1.25% w/w, such as 0.1% to 1.1% w/w, such as 0.2% to 1.0% w/w and including 0.24% to 0.9% w/w. In other embodiments, the amount of water is 0.01% by weight or greater of the total weight of the transdermal composition, such as 0.025% by weight or greater, such as 0.05% by weight or greater, such as 0.1% by weight or greater, such as 0.25% by weight or greater and including 0.9% by weight water or greater of the total weight of the transdermal composition.

The size of subject transdermal delivery devices may vary, and in some instances the devices are sized to cover the entire application site on the subject. As such, the transdermal delivery device may have a length ranging from 2 to 100 cm, such as from 4 to 60 cm and a width ranging from 2 to 100 cm, such as from 4 to 60 cm. As such, the area of the transdermal delivery device may range from 4 cm² to 10,000 cm², such as from 5 cm² to 1000 cm², such as from 10 cm² to 100 cm², such as from 15 cm² to 50 cm² and including from 20 cm² to 40 cm². In certain embodiments, the transdermal delivery device is sized to have an area of 25 cm². In certain instances, the transdermal delivery device is insoluble in water. By insoluble in water is meant that that the transdermal delivery device may be immersed in water for a period of 1 day or longer, such as 1 week or longer, including 1 month or longer, and exhibit little if any dissolution, e.g., no observable dissolution. In other words, the transdermal delivery device is water-resistant and will not change or be altered in any way when in contact with water.

In certain embodiments, the transdermal delivery device as described above furthers includes a backing layer. The backing may be flexible, such that it can be brought into close contact with the desired application site on the subject. The backing may be fabricated from a material that it does not absorb the tropane compound or pharmaceutically acceptable salt thereof, and does not allow the tropane compound or pharmaceutically acceptable salt thereof to be leached from the active agent layer. Backing layers of interest may include, but is not limited to, non-woven fabrics, woven fabrics, films (including sheets), porous bodies, foamed bodies, paper, composite materials obtained by laminating a film on a non-woven fabric or woven fabric, and combinations thereof.

Non-woven and woven fabric may include polyolefin resins such as polyethylene and polypropylene; polyester resins such as polyethylene terephthalate, polybutylene terephthalate and polyethylene naphthalate; rayon, polyamide, poly(ester ether), polyurethane, polyacrylic resins, polyvinyl alcohol, styrene-isoprene-styrene copolymers, and styrene-ethylene-propylene-styrene copolymers; and combinations thereof. Fabrics may include cotton, rayon, polyacrylic resins, polyester resins, polyvinyl alcohol, and combinations thereof. Films may include polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof. Papers may include impregnated paper, coated paper, wood free paper, Kraft paper, Japanese paper, glassine paper, synthetic paper, and combinations thereof. In some embodiments, the backing layer is formed from a heat-resistant, non-conducting material. For example, the backing layer may be formed from a heat insulating fabric which does not conduct externally applied heat.

Depending on the dosage interval and the desired target dosage, the size of the backing may vary, and in some instances sized to cover the entire application site on the subject. As such, the backing layer may have a length ranging from 2 to 100 cm, such as 4 to 60 cm and a width ranging from 2 to 100 cm, such as 4 to 60 cm. In certain instances, the backing layer may insoluble in water. By insoluble in water is meant that that the backing layer may be immersed in water for a period of 1 day or longer, such as 1 week or longer, including 1 month or longer, and exhibit little if any dissolution, e.g., no observable dissolution. In other words, the backing layer is water-resistant and will not change or be altered in any way when in contact with water.

The subject transdermal delivery devices are configured to deliver the tropane active agent by passive transport. By “passive transport” is meant that the transdermal delivery device is configured to deliver the tropane active agent across the skin in the absence of applied energy (e.g., rubbing or heat) and is primarily dependent on the permeability of the barrier (e.g., skin) and by entropy of delivery.

The term “dosage interval” is used herein to refer in its conventional sense to the duration of a single administration of the transdermal delivery device. In other words, a dosage interval begins with applying the transdermal composition to the skin and ends with the removal of the transdermal composition from contact with the skin. As such, a dosage interval may last about 24 hours or longer, such as about 48 hours or longer, such as about 72 hours or longer, such as 96 hours or longer, such as 120 hours or longer, such as 144 hours or longer and including about 168 hours or longer. The term “treatment regimen” as used herein refers to one or more sequential dosage intervals sufficient to produce the desired therapeutic effect of the transdermal composition. Treatment regimens may include one or more dosage intervals, as desired, such as two or more dosage intervals, such as five or more dosage intervals, including ten or more dosage intervals.

In other embodiments, transdermal delivery devices having an amount of the tropane active agent and a pressure sensitive adhesive may be configured to deliver an average cumulative amount of the tropane active agent of 60 μg/cm² or greater over an extended period of time. The term “cumulative amount” refers to the total quantity of the tropane active agent delivered by the transdermal delivery device. In these embodiments, transdermal delivery devices of interest may be configured to deliver an average cumulative amount of the tropane active agent that is 60 μg/cm² or greater, such as 75 μg/cm² or greater, such as 100 μg/cm² or greater over a 7 day delivery interval, such as 125 μg/cm² or greater, such as 150 μg/cm² or greater, such as 175 pg/cm² or greater and including 200 μg/cm² over a predetermined dosage interval.

In certain instances, the subject transdermal delivery devices are configured to deliver an average cumulative amount of the tropane active agent of 60 μg/cm² or greater where the tropane active agent is present in the transdermal composition in an amount which is 10% or less by weight of the total weight of the transdermal composition, such as 8% or less by weight, such as 6% or less by weight and including 4% or less by weight of the total weight of the transdermal composition. For example, in some instances, the transdermal delivery device is configured to deliver an average cumulative amount of tropane active agent ranging from 80 μg/cm²to120 μg/cm² where the tropane active agent is present in the transdermal composition in an amount which is 4% or less by weight. In other instances, the transdermal delivery device is configured to deliver an average cumulative amount of tropane active agent ranging from 150 μg/cm² to 200 μg/cm² where the tropane active agent is present in the transdermal composition in an amount which is 10% or less by weight.

In some embodiments, transdermal delivery devices of interest may include transdermal delivery devices having an amount of the tropane active agent and a pressure sensitive adhesive that are configured in a manner sufficient to deliver a predetermined dosage of the tropane active agent. The term “predetermined dosage” is meant the desired amount of the tropane active agent to be delivered from the transdermal delivery device. For example, transdermal delivery devices may be configured in a manner sufficient to deliver a predetermined dosage of the tropane active agent of 10 μg/hr or greater, such as 20 μg/hr or greater, such as 40 μg/hr or greater, such as 80 μg/hr or greater, such as 120 μg/hr or greater, such as 160 μg/hr or greater, such as 200 μg/hr or greater and including 400 μg/hr or greater. In certain embodiments, the transdermal delivery device may be configured in a manner sufficient to deliver a predetermined dosage of the tropane active agent ranging from 20 to 200 μg/hr, such as 215 to 180 μg/hr, such as 22 to 160 μg/hr, such as 25 to 150 pg/hr, such as 30 to140 μg/hr, such as 35 to 130 μg/hr and including 40 to 120 μg/hr.

Depending on the desired therapeutic effect of the transdermal composition, the subject transdermal delivery devices may be configured to deliver a predetermined dosage of the tropane active agent as desired. In certain embodiments, the predetermined dosage of the tropane active agent in the transdermal delivery devices of interest is an amount which is in the “therapeutic window” of a subject, such as a human being. The term “therapeutic window” is used herein in its conventional sense to refer to the dosage range which is considered to be therapeutically effective for a particular drug. A therapeutically effective amount is an amount that when applied to a subject provides for a desired therapeutic activity.

In some embodiments, transdermal delivery devices are configured to maintain a predetermined mean plasma concentration of the tropane active agent in the subject. For example, the transdermal delivery device is configured, according to certain embodiments, to deliver the tropane active agent to the subject in a manner sufficient to maintain a mean plasma of from about 0.01 ng/mL to about 50.0 ng/mL, such as from about 0.05 ng/mL to about 40.0 ng/mL, such as from about 0.1 ng/mL to about 35 ng/mL, such as from about 0.5 ng/mL to about 30 ng/mL, such as from about 1 ng/mL to about 25 ng/mL and including from about 2 ng/mL to about 20 ng/mL.

The transdermal delivery devices may, in certain instances, be configured to reach peak delivery of the tropane active agent to the subject (i.e., peak transdermal flux) in 10 hours or less, such as in 9 hours or less, such as in 8 hours or less, such as in 7 hours or less, such as in 6 hours or less, such as in 5 hours or less, such as in 4 hours or less, such as in 3 hours or less, such as in 2 hours or less and including being configured to reach peak transdermal flux of the tropane active agent in 1 hour or less. The peak flux of the tropane active agent achieved by the transdermal delivery devices may vary, such as 0.5 μg/cm²/hr or greater, such as 0.6 μg/cm²/hr or greater, such as 0.65 μg/cm²/hr or greater, such as 0.75 μg/cm²/hr, such as 0.9 μg/cm²/hr, such as 1.0 μg/cm²/hr or greater, such as 1.5 μg/cm²/hr or greater, such as 1.75 μg/cm²/hr or greater, such as a peak flux of 2.0 μg/cm²/hr or greater, such as 5 μg/cm²/hr or greater, such as 7 μg/cm²/hr or greater and including a peak flux of 10 μg/cm²/hr or greater.

Transdermal delivery devices of interest may also include devices having an amount of the tropane active agent and a pressure sensitive adhesive that are configured to provide a transdermal flux of the tropane active agent for an extended period of time which is within 90% or greater of peak transdermal flux when measured by an in-vitro protocol, such as for example protocols employing human cadaver skin with epidermal layers (stratum corneum and epidermis) in a Franz cell having donor and receptor sides clamped together and receptor solution containing phosphate buffer. By providing transdermal flux for an extended period of time which is within 90% of peak transdermal flux of the tropane compound or pharmaceutically acceptable salt thereof is meant that the transdermal delivery device is configured to provide a delivery rate of the tropane active agent that does not decrease below 90% of the peak flux after reaching the peak flux as determined by an in-vitro protocol. In other words, the subject transdermal delivery device continues to maintain a constant average flux of the tropane active agent which is within 90% of the peak flux. For example, the transdermal delivery device is configured to maintain a flux of the tropane active agent which is within 95% or more of peak transdermal flux, such as within 96% or more, such as within 97% or more, such as within 98% and including within 99% of peak transdermal flux of the tropane active agent after reaching peak transdermal flux. In certain embodiments, the transdermal delivery device is configured to maintain a flux of the tropane active agent which does not decrease at all after reaching peak flux and maintains a rate of 100% of peak flux. In certain embodiments, transdermal delivery devices of interest include a transdermal composition having an amount of the tropane active agent and an acrylic adhesive, where the extended transdermal delivery device is configured to provide a transdermal flux of the tropane active agent for an extended period of time which is within 90% or greater of peak transdermal flux when measured by an in-vitro protocol. In these embodiments, the transdermal composition may be a single layer matrix incorporating the tropane active agent and acrylic adhesive as well as any additional components into a single layer.

The flux of an active agent by transdermal administration is the rate of penetration of the active agent, such as when determined by an in-vitro protocol, for example by protocols employing human cadaver skin with epidermal layers (stratum corneum and epidermis) in a Franz cell having donor and receptor sides clamped together and receptor solution containing phosphate buffer. For instance, the flux of tropane compound or pharmaceutically acceptable salt thereof can be determined by the equation:

J_{skin flux}=P×C (1)

where J is the skin flux, C is the concentration gradient across the skin and P is the permeability coefficient. Skin flux is the change in cumulative amount of drug passing across the skin with respect to time.

As such, the subject extended transdermal delivery devices are configured to provide a steady state average flux of the tropane compound or pharmaceutically acceptable salt thereof when determined by an in-vitro protocol (e.g., using cadaver skin in a Franz cell). The term “steady state” is used in its conventional sense to mean that transdermal delivery device can deliver a substantially constant amount of the tropane compound or pharmaceutically acceptable salt thereof. By “substantially constant” is meant the flux of the tropane active agent increases or decreases by 10% or less at any time while in use, such as 8% or less, such as 7% or less, such as 6% or less, such as 5% or less, such as 3% or less, such as 2.5% or less, such as 2% or less, and including 1% or less at any time while in use. In other words, extended transdermal delivery devices of interest may be configured in a manner sufficient to deliver a “zero-order” flux of the tropane compound or pharmaceutically acceptable salt thereof after reaching peak flux.

Transdermal delivery devices of interest may also include transdermal delivery devices having an amount of the tropane active agent and a pressure sensitive adhesive that are configured to deliver the tropane active agent at a substantially linear rate over a predetermined dosage interval (e.g., 7 days or longer). By “substantially linearly” is meant that the cumulative amount of the tropane active agent released from the transdermal composition increases at a substantially constant rate (i.e., defined by first-order kinetics). As such, the change in rate of cumulatively delivered tropane active agent increases or decreases by 10% or less at any given time, such as 8% or less, such as 7% or less, such as 6% or less, such as 5% or less, such as 3% or less, such as 2.5% or less, such as 2% or less, and including 1% or less.

In these embodiments, the transdermal delivery device is configured to provide a constant flux, such as by providing an excess of the tropane active agent in the transdermal delivery device. For example, an excess in the tropane active agent may be a 5% or greater excess of the predetermined dosage of the tropane active agent, such as a 10% excess or greater, such as a 15% excess or greater, such as a 20% excess or greater, and including a 25% excess or greater of the predetermined dosage. Where an excess in predetermined dosage of the tropane active agent is employed in order to provide a constant flux, the excess amount is not absorbed during administration of the transdermal delivery device.

In certain embodiments, transdermal delivery devices are configured to deliver the tropane active agent to the subject:

at a substantially second order rate for a first predetermined period of time; and

at a substantially first order rate for a second predetermined period of time.

In these embodiments, the first period may be a duration ranging from 0.5 hours to 72 hours from administering the transdermal delivery device to the subject, such as from 1 hour to 60 hours, such as from 2 hours to 48 hours, such as from 3 hours to 36 hours, such as from 4 hours to 30 hours and including from 5 hours to 24 hours from the time of administration. The second period may be a duration ranging from 0.5 hours to 336 hours from administering the transdermal delivery device to the subject, such as from 1 hour to 312 hours, such as from 2 hours to 288 hours, such as from 3 hours to 264 hours, such as from 4 hours to 240 hours, such as from 5 hours to 216 hours and including from 6 hours to 192 hours from the time of administration. In certain instances, the first period of time is 24 hours or less, such as 20 hours or less, such as 16 hours or less, such as 12 hours or less, such as 8 hours or less and including 4 hours or less. In some instances, the second period of time is 1 hour or more, such as 2 hours or more, such as 4 hours or more, such as 8 hours or more, such as 12 hours or more, such as 16 hours or more, such as 20 hours or more and including 24 hours or more.

In other embodiments, transdermal delivery devices are configured to deliver the tropane active agent to the subject:

at a substantially first order rate for a first predetermined period of time; and

at a substantially zero order rate for a second predetermined period of time.

In these embodiments, the first period may be a duration ranging from 0.5 hours to 72 hours from administering the transdermal delivery device to the subject, such as from 1 hour to 60 hours, such as from 2 hours to 48 hours, such as from 3 hours to 36 hours, such as from 4 hours to 30 hours and including from 5 hours to 24 hours from the time of administration. The second period may be a duration ranging from 0.5 hours to 336 hours from administering the transdermal delivery device to the subject, such as from 1 hour to 312 hours, such as from 2 hours to 288 hours, such as from 3 hours to 264 hours, such as from 4 hours to 240 hours, such as from 5 hours to 216 hours and including from 6 hours to 192 hours from the time of administration. In certain instances, the first period of time is 24 hours or less, such as 20 hours or less, such as 16 hours or less, such as 12 hours or less, such as 8 hours or less and including 4 hours or less. In some instances, the second period of time is 1 hour or more, such as 2 hours or more, such as 4 hours or more, such as 8 hours or more, such as 12 hours or more, such as 16 hours or more, such as 20 hours or more and including 24 hours or more.

### Methods for Applying to a Subject Transdermal Delivery Devices Containing a Tropane Active Agent

Aspects of the invention include methods of treating a subject with the subject transdermal delivery devices having a tropane active agent. In practicing the subject methods according to certain embodiments, a transdermal delivery device having a tropane active agent as detailed above is applied to a skin surface of a subject and maintained in contact with the subject for an amount of time sufficient to deliver a therapeutically effective amount of the tropane active agent to the subject. As such, methods according to embodiments are sufficient to deliver to the subject a therapeutically effective amount of a tropane active agent.

In embodiments, the tropane active agent is delivered across the skin (e.g., topical administration) for systemic distribution. As such, transdermal compositions as described herein include compositions which are delivered to the subject through one or more of the subcutis, dermis and epidermis, including the stratum corneum, stratum germinativum, stratum spinosum and stratum basale. Accordingly, transdermal delivery devices containing the tropane active agent may be applied at any convenient location, such as for example, the arms, legs, buttocks, abdomen, back, neck, scrotum, face, behind the ear, etc. In describing methods of the present invention, the term “subject” is meant the person or organism to which the transdermal composition is applied and maintained in contact. As such, subjects of the invention may include but are not limited to mammals, e.g., humans and other primates, such as chimpanzees and other apes and monkey species; and the like, where in certain embodiments the subject are humans. The term subject is also meant to include a person or organism of any age, weight or other physical characteristic, where the subjects may be an adult, a child, an infant or a newborn.

Transdermal administration of the tropane compound or pharmaceutically acceptable salt thereof may be passive. By “passive” transport is meant that the tropane active agent is delivered across the skin in the absence of applied energy (e.g., rubbing or heat) and is primarily dependent on the permeability of the barrier (e.g., skin) and by entropy of delivery. The subject methods may be suitable for treating a subject having conditions or maladies associated with one or more of serotonin, norepinephrine or dopamine reuptake activity.

In certain embodiments, methods include treating a subject for cocaine addiction with the transdermal delivery devices described herein. In one embodiment, the transdermal device as described herein is for use in a method of treating cocaine addiction, i.e. the transdermal device is for use in treating cocaine addiction. In one embodiment, the present disclosure relates to use of a transdermal delivery device as described herein in the manufacture of a medicament for treatment of cocaine addiction. In certain of these embodiments of the invention, methods include applying a transdermal delivery device having a composition that includes the tropane active agent NS2359 or a pharmaceutically acceptable salt thereof to a subject and maintaining the transdermal delivery device in contact with the subject in a manner sufficient to deliver an amount of the tropane active agent to the subject. In one embodiment, methods include applying a transdermal delivery device having a composition that includes the tropane active agent NS2359, or a pharmaceutically acceptable salt thereof, to a subject, treating said subject for cocaine addiction.

In certain embodiments, the subject is a person that has been diagnosed as having cocaine addiction and the subject methods are sufficient to treat the subject for cocaine addiction. The term “cocaine addiction” is used in its conventional sense to the dependence of the subject on cocaine. Cocaine addiction may be the physiological dependence on cocaine, such as where the body has adjusted to incorporate cocaine into normal functioning resulting in increased tolerance and experiences of withdrawal by the subject. Likewise, cocaine addiction may also be psychological, such as where dependence on cocaine is a behavioral addiction. In some embodiments, the subject methods are sufficient to treat the subject for physiological dependence on cocaine. In other embodiments, the subject methods are sufficient to treat the subject for psychological dependence on cocaine. In still other embodiments, the subject methods are sufficient to treat the subject for physiological and psychological dependence on cocaine.

In certain embodiments, methods include cocaine detoxification. The term “cocaine detoxification” is used in its conventional sense to refer to the physiological or medicinal removal of cocaine or cocaine metabolites in the subject and includes the period of withdrawal during which the subject returns to homeostasis. Depending on the physiology of the subject, age and course of treatment as determined by a qualified health care or drug intervention professional, transdermal delivery devices of interest may be employed in an cocaine detoxification protocol, which may include, but is not limited to chronic medical detoxification, acute medical detoxification, rapid detoxification, stepped rapid detoxification, ultra-rapid detoxification, as well as interval (e.g., outpatient) detoxification. In practicing methods of cocaine detoxification according to certain instances, one or more transdermal delivery devices having a tropane compound or pharmaceutically acceptable salt thereof are applied to a subject and maintained in contact with the subject in a manner sufficient to deliver an amount of the tropane active agent to carry out cocaine detoxification in the subject.

By “treating cocaine addiction” or “treatment of cocaine addiction” is meant at least a suppression or amelioration of the symptoms associated with the subject's dependence (e.g., physiological or psychological) on cocaine, where suppression and amelioration are used in a broad sense to refer to at least a reduction in the magnitude of a parameter, e.g., symptom, associated with the cocaine addiction. As such, treatment of cocaine addiction includes situations where the cocaine addiction is completely stopped, e.g., terminated, such that the subject no longer experiences cocaine addiction.

Transdermal devices as described herein are useful as a medicament, e.g. for the treatment, prevention or alleviation of obesity and/or an obesity associated disorder. In certain such instances, the tropane active agent is Tesofensine or a pharmaceutically acceptable salt thereof. In one embodiment, the present disclosure relates to a transdermal delivery device as described herein for use in the treatment of obesity or an obesity related disorder. In one embodiment, the present disclosure relates to the use of a transdermal delivery device as described herein for in the manufacture of a medicament for treatment of obesity or an obesity related disorder. In one embodiment, the transdermal delivery device having a composition that includes the tropane active agent Tesofensine, or a pharmaceutically acceptable salt thereof, is applied to a subject in a method of treating said subject for obesity or an obesity related disorder.

In one embodiment transdermal devices of the present disclosure are for use in the treatment of obesity.

Obesity is defined herein as a medical condition in which excess body fat has accumulated to the extent that it may have an adverse effect on health, leading to reduced life expectancy and/or increased health problems in general. Thus, in one embodiment the subject to be treated with the devices of the present disclosure is obese.

Body mass index (BMI) is a measure which compares weight and height. People are generally considered overweight or pre-obese if the BMI is between 25 and 30 and obese if the BMI is over 30. Morbidly obese subjects have a BMI over 35.

In one embodiment the subject has a BMI above 25 kg/m², such as above 30 kg/m², for example above 35 kg/m², such as above 40 kg/m².

In one embodiment the subject has a BMI above 30 kg/m².

In one embodiment the subject has a BMI above 35 kg/m².

In one embodiment devices of the present disclosure are for use in the treatment of an obesity associated disorder, such as a disease or disorder selected from the group consisting of diabetes, metabolic syndrome, dyslipidemia, atherosclerosis, drug-induced obesity, overeating disorders, bulimia nervosa, binge eating disorder, compulsive over-eating, impaired appetite regulation, nonalcoholic fatty liver disease (NAFLD) and nonalcoholic steatohepatitis (NASH).

In one embodiment devices of the present disclosure are for use in the treatment of diabetes, such as type 1 diabetes, type 2 diabetes, prediabetes and gestational diabetes. Preferably, the diabetic subject is obese.

In one embodiment devices of the present disclosure are for use in the treatment of type 1 diabetes.

In one embodiment devices of the present disclosure are for use in the treatment of type 2 diabetes.

In one embodiment devices of the present disclosure are for use in the treatment of prediabetes.

In one embodiment devices as described herein lead to an alleviation or improvement of diabetic complications.

Type 1 diabetes (diabetes mellitus type 1) is a form of diabetes that results from the autoimmune destruction of the insulin-producing beta cells in the pancreas. In type 1 diabetes, hypertension may reflect the onset of diabetic nephropathy.

Type 2 diabetes is a metabolic disorder that is characterized by hyperglycemia in the context of insulin resistance and a relative lack of insulin. Type 2 diabetes makes up about 90% of cases of diabetes, with the other 10% due primarily to diabetes mellitus type 1 and gestational diabetes. Obesity is thought to be the primary cause of type 2 diabetes in people who are genetically predisposed to the disease. Pre-diabetes is used interchangeably herein with intermediate hyperglycaemia.

Intermediate hyperglycaemia is a biochemical state in which a person has glucose levels above the normal range, but does not yet meet the criteria for a diagnosis of diabetes. The primary aim of management of intermediate hyperglycaemia is to prevent progression to diabetes.

A pre-diabetic subject may have one or more of impaired fasting glycaemia (IFG) and/or impaired glucose tolerance (IGT) and/or elevated glycated haemoglobin (HbAic) levels.

Weight loss can prevent progression of pre-diabetes into diabetes and can also markedly improve clinical symptoms of type 2 diabetes. Thus, weight loss is an attractive treatment strategy for pre-diabetic subjects and subjects suffering from type 2 diabetes.

In one embodiment the subject is an obese, pre-diabetic human. In one embodiment the subject is an obese subject suffering from type 2 diabetes.

Gestational diabetes is a condition in which women without previously diagnosed diabetes exhibit high blood glucose levels during pregnancy (especially during their third trimester). Gestational diabetes is caused when insulin receptors do not function properly.

The WHO diabetes diagnostic criteria are shown in the table below.

| | | | | | HbA_{1c} | |
|---|---|---|---|---|---|---|
| | 2 hour glucose* | | Fasting glucose | | mmol/mol | |
| Condition | mmol/l (mg/dl) | | mmol/l (mg/dl) | | (DCCT %) | |
| Normal | <7.8 | (<140) | <6.1 | (<110) | <42 | (<6.0) |
| Impaired fasting | <7.8 | (<140) | ≥6.1(≥110) & | | 42-46 | (6.0-6.4) |
| glycemia | | | <7.0(<126) | | | |
| Impaired glucose | ≥7.8 | (≥140) | <7.0 | (<126) | 42-46 | (6.0-6.4) |
| tolerance | | | | | | |
| Diabetes mellitus | ≥11.1 | (≥200) | ≥7.0 | (≥126) | ≥48 | (≥6.5) |

*Venous plasma glucose 2 hours after ingestion of 75 g oral glucose load

The subject benefitting from treatment with devices of the present disclosure may also be a subject suffering from an obesity-associated disorder or condition, such as one selected from the group consisting of diabetes, metabolic syndrome, dyslipidemia, atherosclerosis, drug-induced obesity, overeating disorders, bulimia nervosa, binge eating disorder, compulsive over-eating, impaired appetite regulation, nonalcoholic fatty liver disease (NAFLD) and nonalcoholic steatohepatitis (NASH).

In one embodiment devices of the present disclosure are for use in the treatment of metabolic syndrome, such as for the treatment of an obese subject suffering from metabolic syndrome.

In one embodiment devices of the present disclosure are for use in the treatment of fatty liver disease, such as nonalcoholic fatty liver disease (NAFLD) or nonalcoholic steatohepatitis (NASH). The subject suffering from NAFLD or NASH is preferably obese.

In one embodiment, devices of the present disclosure are for use in the treatment of nonalcoholic fatty liver disease (NAFLD).

In one embodiment, devices of the present disclosure are for use in the treatment of nonalcoholic steatohepatitis (NASH).

Nonalcoholic fatty liver disease (NAFLD) is a cause of a fatty liver, occurring when fat is deposited in the liver (steatosis) due to other causes than excessive alcohol use. NAFLD is the most common liver disorder in Western industrialized nations. NAFLD is associated with insulin resistance and metabolic syndrome (obesity, combined hyperlipidemia, diabetes mellitus (type II) and high blood pressure). Non-alcoholic steatohepatitis (NASH) is the most extreme form of NAFLD, and is a major cause of cirrhosis of the liver. NASH is a state in which the steatosis is combined with inflammation and fibrosis (steatohepatitis).

In one embodiment devices of the present disclosure are for use in a method of decreasing liver fat and/or visceral adiposity. Reduction of liver fat and/or visceral adiposity has been shown to be effective in the treatment of fatty liver disorders. Tesofensine significantly decreases waist circumference and sagittal diameter (Astrup et al., 2008, Lancet 372: 1906-13); hence tesofensine is capable of reducing visceral adiposity.

In one embodiment the present disclosure relates to use of devices as disclosed herein in the manufacture of a medicament for the treatment of diabetes, obesity or an obesity associated disorder.

In some embodiments, methods include transdermal extended delivery of the tropane active agent to the subject. The term “extended delivery” is used herein to refer to a transdermal delivery device that includes a composition formulated to deliver the tropane active agent over an extended period of time, such as over the course of hours, days and including weeks, including 1 hour or longer, such as 2 hours or longer, such as 4 hours or longer, such as 8 hours or longer, such as 12 hours or longer, such as 24 hours or longer, such as 48 hours or longer, such as 72 hours or longer, such as 96 hours or longer, such as 120 hours or longer, such as 144 hours or longer and including 168 hours or longer. For the above ranges an upper limit period of time is, in some instances, 168 hours or shorter, such as 144 hours or shorter, such as 120 hours or shorter, such as 96 hours or shorter, such as 72 hours or shorter, such as 48 hours or shorter and including 24 hours or shorter. In certain embodiments, extended transdermal delivery ranges such as from 0.5 hours to 168 hours, such as from 1 hour to 144 hours, such as from 1.5 hours to 120 hours, such from 2 hours to 96 hours, such as from 2.5 hours to 72 hours, such as from 3 hours to 48 hours, such as from 3.5 hours to 24 hours, such as from 4 hours to 12 hours and including from 5 hours to 8 hours.

In some embodiments, methods include multi-day delivery of a therapeutically effective amount of the tropane compound to the subject. By multi-day delivery is meant that the transdermal composition is formulated to provide a therapeutically effective amount to a subject when the transdermal delivery device is applied to the skin of a subject for a period of time that is 1 day or longer, such as 2 days or longer, such as 4 days or longer, such as 7 days or longer, such as 14 days and including 30 days or longer. In certain embodiments, transdermal delivery devices provide a therapeutically effective amount of the tropane compound to a subject for a period of 10 days or longer. For multi-day delivery, an upper limit period of time is, in some instances, 30 days or shorter, such as 28 days or shorter, such as 21 days or shorter, such as 14 days or shorter, such as 7 days or shorter and including 3 days or shorter. In certain embodiments, multi-day transdermal delivery ranges such as from 2 days to 30 days, such as from 3 days to 28 days, such as from 4 days to 21 days, such as from 5 days to 14 days and including from 6 days to 10 days.

Depending on the specific protocol employed, methods according to embodiments of the present disclosure may include one or more treatment dosage intervals. The term “dosage interval” is used herein in its conventional sense to mean the duration of a single administration of applying and maintaining the transdermal delivery device in contact with the subject. In other words, a dosage interval begins with applying the transdermal delivery device to the skin of the subject and ends with the removal of the transdermal delivery device from contact with the subject. As such, a dosage interval is the period of time that an amount of the tropane compound or pharmaceutically acceptable salt thereof is in contact with the skin of the subject and may last about 0.5 hours or longer, such as 1 hour or longer, such as 2 hours or longer, such as 4 hours or longer, such as 8 hours or longer, such as 12 hours or longer, such as 16 hours or longer, such as 20 hours or longer, such as 24 hours or longer, such as about 48 hours or longer, such as about 72 hours or longer, such as 96 hours or longer, such as 120 hours or longer, such as 144 hours or longer and including about 168 hours or longer. An upper limit period of time for the duration of dosage intervals is, in some instances, 168 hours or shorter, such as 144 hours or shorter, such as 120 hours or shorter, such as 96 hours or shorter, such as 72 hours or shorter, such as 48 hours or shorter and including 24 hours or shorter. In certain embodiments, the duration of dosage intervals ranges such as from 0.5 hours to 168 hours, such as from 1 hour to 144 hours, such as from 1.5 hours to 120 hours, such from 2 hours to 96 hours, such as from 2.5 hours to 72 hours, such as from 3 hours to 48 hours, such as from 3.5 hours to 24 hours, such as from 4 hours to 12 hours and including from 5 hours to 8 hours.

The term “treatment protocol” as used herein refers to one or more sequential dosage intervals sufficient to produce the desired therapeutic effect of the tropane active agent. In certain embodiments, protocols may include multiple dosage intervals. By “multiple dosage intervals” is meant more than one transdermal delivery device is applied and maintained in contact with the subject in a sequential manner. As such, a transdermal delivery device is removed from contact with the subject and a new transdermal delivery device is reapplied to the subject. In practicing methods of the invention, treatment regimens may include two or more dosage intervals, such as three or more dosage intervals, such as four or more dosage intervals, such as five or more dosage intervals, including ten or more dosage intervals.

The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 3 days or longer, such as 4 days or longer, such as 5 days or longer, such as 6 days or longer, such as 7 days or longer, such as 10 days or longer, including 30 days or longer. An upper limit period of time between dosage intervals is, in some instances, 30 days or shorter, such as 28 days or shorter, such as 21 days or shorter, such as 14 days or shorter, such as 7 days or shorter and including 3 days or shorter. In certain embodiments, the time between dosage intervals ranges such as from 2 days to 30 days, such as from 3 days to 28 days, such as from 4 days to 21 days, such as from 5 days to 14 days and including from 6 days to 10 days.

In certain instances, the duration between dosage intervals may depend on the plasma concentration of the tropane active agent during the time the transdermal delivery device is not in contact with the subject between dosage intervals. For example, a subsequent dosage interval may commence when the plasma concentration of the tropane active agent reaches below a particular threshold.

As described above, aspects of the disclosure include treating the subject by applying a transdermal delivery device containing a tropane active agent. In some embodiments, methods include maintaining the transdermal delivery device in contact with a subject in a manner sufficient to deliver a target dosage of the tropane active agent to the subject, such as for example delivering a target dosage as determined by total drug exposure or by average daily drug exposure. The term target dosage is meant the desired amount of permeated tropane active agent. Depending on the desired therapeutic effect of the tropane active agent, the treatment protocol and the physiology of the subject, target drug exposure of the tropane active agent may vary. In certain embodiments, the target drug exposure is an amount in the therapeutic window of the subject. The term “therapeutic window” is used herein to refer to the dosage range which is therapeutically effective in treating a subject. For example, the target dosage of the tropane active agent may range from 50 μg/day to 5000 μg/day, such as from 100 μg/day to 4500 μg/day, such as from 145 μg/day to 4000 μg/day, such as from 155 μg/day to 3500 μg/day, such as from 165 μg/day to 3000 μg/day, such as from 175 μg/day to 2500 μg/day, such as from 195 μg/day to 2000 μg/day and including from 200 μg/day to 1500 μg/day over the course of a dosage interval (e.g., a 168 hour dosage interval). In certain embodiments, the target dosage of the tropane active agent ranges from 145 μg/day to 5000 μg/day over the course of a dosage interval (e.g., a 168 hour or longer dosage interval).

In some embodiments, the target dosage is an amount that when applied to a subject provides for a systemic amount of the tropane active agent that gives a desired mean plasma concentration of the tropane active agent at specific times during treatment. In other embodiments, the target dosage is an amount that when applied to a subject provides for a steady state mean plasma concentration of the tropane active agent throughout a dosage interval or treatment protocol. In other embodiments, the target dosage is an amount that when applied to a subject provides for a particular rate of delivery of the tropane active agent to the subject in vivo.

In some embodiments, applying and maintaining a transdermal delivery device containing the tropane active agent in contact with a subject includes delivery of a target amount of the tropane active agent, such as for example an average cumulative amount of the tropane active agent delivered over the course of a dosage interval (e.g., 7 days or longer.

Methods according to certain embodiments may include applying to the subject a transdermal delivery device containing an amount of the tropane active agent and maintaining the transdermal composition in contact with the subject in a manner sufficient to provide a mean plasma concentration which ranges from about 0.001 ng/mL to about 50 ng/mL, such as from about 0.005 ng/mL to about 45 ng/mL, such as from about 0.01 ng/mL to about 40 ng/mL, such as from about 0.01 ng/mL to about 35 ng/mL, such as from about 0.05 ng/mL to about 30ng/mL and including from about 0.01 ng/mL to about 25 ng/mL. For example, the transdermal delivery device may be maintained in contact with the subject in a manner sufficient to provide a mean plasma concentration which ranges from 0.01 ng/mL to 50 ng/mL over the course of a dosage interval. In other embodiments, methods include maintaining the transdermal composition in contact with the subject in a manner sufficient to provide a mean plasma concentration which ranges from 0.001 ng/mL to 50 ng/mL over the course of the entire treatment protocol (i.e., over one or more dosage intervals), such as from about 0.005 ng/mL to about 45 ng/mL, such as from about 0.01 ng/mL to about 40 ng/mL, such as from about 0.01 ng/mL to about 35 ng/mL, such as from about 0.05 ng/mL to about 30 ng/mL and including from about 0.01 ng/mL to about 25 ng/mL over the course of the entire treatment protocol. For example, the transdermal delivery device may be maintained in contact with the subject in a manner sufficient to provide a mean plasma concentration which ranges from 0.01 ng/mL to 50 ng/mL over the course of the entire treatment protocol.

In certain embodiments, methods may also include determining the plasma concentration of the tropane active agent in the subject. The plasma concentration may be determined using any convenient protocol, such for example by liquid chromatography-mass spectrometry (LCMS). The plasma concentration of the tropane active agent may be determined at any time desired. In some embodiments, the plasma concentration of the tropane active agent may be monitored throughout the entire time the transdermal delivery device is maintained in contact with the subject, such by real-time data collection. In other instances, the plasma concentration of the tropane active agent is monitored while maintaining the transdermal delivery device in contact with the subject by collecting data at regular intervals, e.g., collecting data every 0.25 hours, every 0.5 hours, every 1 hour, every 2 hours, every 4 hours, every 12 hours, every 24 hours, including every 72 hours, or some other interval. In yet other instances, the plasma concentration of the tropane active agent is monitored while maintaining the transdermal delivery device in contact with the subject by collecting data according to a particular time schedule after applying the transdermal delivery device to the subject. For instance, the plasma concentration of the tropane active agent may be determined 15 minutes after applying the transdermal delivery device to the subject, 30 minutes after applying the transdermal delivery device to the subject, 1 hour after applying the transdermal delivery device to the subject, 2 hours after applying the transdermal delivery device to the subject, 4 hours after applying the transdermal delivery device to the subject, 8 hours after applying the transdermal delivery device to the subject, 12 hours after applying the transdermal delivery device to the subject, 24 hours after applying the transdermal delivery device to the subject, 48 hours after applying the transdermal delivery device to the subject, 72 hours after applying the transdermal delivery device to the subject, 76 hours after applying the transdermal delivery device to the subject, 80 hours after applying the transdermal delivery device to the subject, 84 hours after applying the transdermal delivery device to the subject, 96 hours after applying the transdermal delivery device to the subject, 120 hours after applying the transdermal delivery device to the subject and including 168 hours after applying the transdermal delivery device to the subject.

In certain embodiments, the plasma concentration of the tropane active agent is determined before the transdermal delivery device is applied to a subject, such as for example, to determine the basal plasma concentration of the tropane active agent. For example, the plasma concentration may be determined 5 minutes before applying the transdermal delivery device, such as 10 minutes before, such as 30 minutes before, such as 60 minutes before, such as 120 minutes before, such as 240 minutes before and including 480 minutes before applying the transdermal delivery device. As described detail below, methods may include multiple dosage intervals where applying and maintaining the transdermal delivery device in contact with the subject may be repeated. In these embodiments, the plasma concentration may be determined after a first transdermal delivery device is removed and before a second transdermal delivery device is applied.

The blood plasma concentration of the tropane active agent may be determined one or more times at any given measurement period, such as 2 or more times, such as 3 or more times, including 5 or more times at each measurement period. An upper limit for the number of times the blood plasma concentration of the tropane active agent is determined at any given measurement period is, in some instances, 10 times or fewer, such as 7 times or fewer, such as 5 times or fewer, such as 3 times or fewer and including 2 times or fewer. In certain embodiments, the number of times the blood plasma concentration of the tropane active agent is determined at any given measurement period ranges such as from 2 times to 10 times, such as from 3 times to 9 times, such as from 4 times to 8 times and including from 5 times to 7 times.

Methods according to certain embodiments may include applying to the subject a transdermal delivery device containing a tropane active agent and maintaining the transdermal composition in contact with the subject in a manner sufficient to maintain a transdermal flux of the tropane active agent thereof which is within 30% or more of the peak transdermal flux of the tropane active agent after reaching the peak transdermal flux. As such, once transdermal delivery devices of interest reach peak transdermal flux of the tropane active agent, the transdermal delivery device is configured to maintain a flux of to the subject that is at least 30% of peak flux of the tropane active agent during the course of any given dosage interval, such as at least 35%, such as at least 40% and including at least 50% of peak flux during the course of any given dosage interval. In other words, once peak flux is reached by the transdermal delivery device according to these particular embodiments, the transdermal flux of the tropane active agent to the subject does not fall below 30% or more of the peak flux at any time during the dosage interval.

For example, the transdermal delivery device may be maintained in contact with the subject in a manner sufficient to maintain the transdermal flux of the tropane active agent which is within 80% or more of peak transdermal flux, such as within 85% or more, such as within 90% or more, such as within 95% and including within 99% of peak transdermal flux of the tropane active agent after reaching peak transdermal flux. In certain embodiments, the transdermal flux does not decrease at all after reaching peak flux of the tropane active agent and maintains a rate of 100% of peak flux from the moment it reaches peak flux of the tropane active agent until the end of a given dosage interval.

In some instances, the transdermal delivery device is maintained in contact with the subject in a manner sufficient to provide a peak flux of the tropane active agent of 0.05 μg/cm²/hr or greater, such as 0.1 μg/cm²/hr or greater, such as 0.5 μg/cm²/hr or greater, such as 1 μg/cm²/hr, such as 2 μg/cm²/hr, such as 3 μg/cm²/hr or greater, such as 5 μg/cm²/hr or greater, such as 7.5 μg/cm²/hr or greater and including maintaining the transdermal delivery device in contact with the subject in a manner sufficient to provide a peak flux of the tropane active agent of 10 μg/cm²/hr or greater. For peak flux of the tropane active agent, an upper limit is, in some instances, 10 μg/cm²/hr or less, such as 9 μg/cm²/hr or less, such as 8 μg/cm²/hr or less, such as 7 μg/cm²/hr or less, 6 μg/cm²/hr or less, such as 5 μg/cm²/hr or less and including 2 μg/cm²/hr or less. In certain embodiments, the peak flux of the tropane active agent ranges such as from 0.05 μg/cm²/hr to 10 μg/cm²/hr, such as from 1 μg/cm²/hr to 9 μg/cm²/hr and including from 2 μg/cm²/hr to 8 μg/cm²/hr.

As such, where the transdermal delivery device is maintained in contact with the subject in a manner sufficient to provide a transdermal flux of the tropane active agent which is within at least 30% of peak transdermal flux, the transdermal composition may be maintained in contact with the subject in a manner sufficient to provide a flux of the tropane active agent which is 0.15 μg/cm²/hr or greater after reaching a peak transdermal flux of 0.5 μg/cm²/hr, such as 0.18 μg/cm²/hr or greater after reaching a peak transdermal flux of 0.6 μg/cm²/hr, such as 0.225 μg/cm²/hr or greater after reaching a peak transdermal flux of 0.75 μg/cm²/hr, such as 0.27 pg/cm²/hr or greater after reaching a peak flux of 0.9 μg/cm²/hr, such as 0.3 μg/cm2/hr or greater after reaching a peak flux of 1.0 μg/cm²/hr, such as 1.5 μg/cm²/hr after reaching a peak flux of 5 μg/cm²/hr or greater and including maintaining the transdermal delivery device in contact with the subject in a manner sufficient to provide a flux of the tropane active agent which is 3.0 μg/cm²/hr or greater after reaching a peak flux of 10.0 μg/cm²/hr.

Depending on the amount of the tropane active agent present in the transdermal composition, the physiology of the subject, target site of application, the time required to reach peak flux may vary. In some instances, peak flux of the tropane active agent is reached 2 hours or more after applying the transdermal delivery device to the subject, such as 4 hours or more, such as 6 hours or more, such as 12 hours or more, such as 18 hours or more and including at 24 hours or more after applying the transdermal delivery device to the subject. In other instances, the peak flux of the tropane active agent is reached at 168 hours or earlier, such as 144 hours or earlier, such as 120 hours or earlier, such as 96 hours or earlier, such as 72 hours or earlier, such as 48 hours or earlier, such as 24 hours or earlier, such as 12 hours or earlier, such as 8 hours earlier, such as 4 hours or earlier and including at 2 hours or earlier. In some embodiments, peak flux of the tropane active agent is reached at 24 hours after applying the transdermal delivery device to the subject.

In certain embodiments, the subject the transdermal delivery devices are maintained in contact with the subject sufficient to provide a steady state average flux of the tropane active agent to the subject. The term “steady state” is used in its conventional sense to mean that the amount of the tropane active agent released maintains a substantially constant average flux of the tropane active agent. As such, the flux of the tropane active agent from transdermal delivery devices of interest increases or decreases by 30% or less at any time while the transdermal delivery device is maintained in contact with the subject, such as 20% or less, such as 15% or less, such as 12% or less, such as 10% or less, such as 6% or less, such as 5% or less, such as 4% or less, and including 1% or less at any time while the transdermal delivery device is maintained in contact with the subject.

Where the transdermal delivery device is maintained in contact with the subject sufficient to provide a steady state average flux of the tropane active agent, the steady state average flux of the tropane active agent may be maintained from for 0.5 hours or longer, such as 1 hour or longer, such as 2 hours or longer, such as 3 hours or longer, such as 4 hours or longer, such as 8 hours or longer, 12 hours or longer, such as 24 hours or longer, such as 36 hours or longer, such as 48 hours or longer, such as 72 hours or longer, such as 96 hours or longer, such as 120 hours or longer, such as 144 hours or longer and including 168 hours or longer. For maintaining a steady state average flux of the tropane active agent, an upper limit is, in some instances, for 168 hours or shorter, such as 144 hours or shorter, such as 120 hours or shorter, such as 96 hours or shorter, such as 72 hours or shorter, such as 48 hours or shorter, such as 24 hours or shorter, such as 12 hours or shorter, such as 8 hours or shorter, such as 4 hours or shorter and including 2 hours or shorter.

In these embodiments, the transdermal delivery device is configured to provide a constant flux, such as by introducing a concentration gradient across the skin or providing an excess in dosage amount of the tropane active agent. For example, transdermal delivery devices of interest may include a dosage of the tropane active agent that is 5% or greater in excess of the normal dosage amount, such as 10% or greater, such as 15% or greater, such as 20% or greater, and including 25% or greater in excess of the normal dosage amount. For the excess tropane active agent present in the transdermal delivery device to provide a constant flux, an upper limit is, in some instances 50% or less in excess, such as 45% or less in excess, such as 25% or less in excess, such as 20% or less in excess and including 10% or less in excess of the normal dosage amount. While transdermal compositions of interest may include an excess in order to provide a constant flux, the excess dosage amount is not absorbed as part of the dosage interval. As such, in some embodiments where the transdermal delivery device is maintained in a manner sufficient to provide a constant flux, 25% or less of the available tropane active agent in the transdermal composition may not be utilized, such as 20% or less, such as 15% or less, such as 10% or less, such as 5% or less and including 1% or less of the available tropane active agent may not be utilized during the dosage interval.

Methods may include applying to the subject a transdermal delivery device containing the tropane active agent and maintaining the transdermal delivery device in contact with the subject in a manner sufficient to provide an average flux of the tropane active agent in vivo of from about 0.005 to about 5 μg/cm².hr, such as from about 0.01 to about 4 μg/cm².hr, such as from about 0.02 to about 3 μg/cm².hr, such as from about 0.05 to about 2.5 μg/cm².hr, such as from about 0.1 to about 2 μg/cm².hr and including from about 0.1 to about 1μg/cm².hr at any time after applying the transdermal delivery device. In some embodiments, methods include applying the transdermal delivery device to the subject and maintaining the transdermal delivery device in contact with the subject in a manner sufficient to provide an average flux of the tropane active agent in vivo of from about 0.005 to about 2.0 μg/cm².hr at 24 hours after application, such as from about 0.01 to about 1.75 μg/cm².hr, such as from about 0.02 to about 1.5 μg/cm².hr, such as from about 0.05 to about 1.25 μg/cm².hr and including from about 0.1 to about 1 μg/cm².hr at 24 hours after application. In yet other embodiments, methods include applying the transdermal delivery device to the subject and maintaining the transdermal composition in contact with the subject in a manner sufficient to provide an average flux of the tropane active agent in vivo of from about 0.005 to about 2.0 μg/cm².hr at 168 hours after application, such as from about 0.01 to about 1.75 μg/cm².hr, such as from about 0.02 to about 1.5 μg/cm².hr, such as from about 0.05 to about 1.25 μg/cm².hr and including from about 0.1 to about 1 μg/cm².hr at 168 hours after application.

In certain embodiments, methods include determining the transdermal flux of the tropane active agent. The transdermal flux may be determined using any convenient protocol, such for example by protocols employing human cadaver skin with epidermal layers (stratum corneum and epidermis) in a Franz cell having donor and receptor sides clamped together and receptor solution containing phosphate buffer. The amount of the tropane active agent that is permeated can further be characterized by liquid chromatography. The transdermal flux may be determined at any time during methods of the present disclosure. In some embodiments, the transdermal flux of the tropane active agent may be monitored throughout the entire time the transdermal delivery device is maintained in contact with the permeation barrier (e.g., human cadaver skin), such by real-time data collection. In other instances, the transdermal flux of the tropane active agent is monitored by collecting data at regular intervals, e.g., collecting data every 0.25 hours, every 0.5 hours, every 1 hour, every 2 hours, every 4 hours, every 12 hours, every 24 hours, including every 72 hours, or some other regular or irregular intervals. In yet other instances, the transdermal flux of the tropane active agent is monitored by collecting data according to a particular time schedule. For instance, the transdermal flux of the tropane active agent may be determined 15 minutes after applying the transdermal delivery device, 30 minutes after applying the transdermal delivery device, 1 hour after applying the transdermal delivery device, 2 hours after applying the transdermal delivery device, 4 hours after applying the transdermal delivery device, 8 hours after applying the transdermal delivery device, 12 hours after applying the transdermal delivery device, 24 hours after applying the transdermal delivery device, 48 hours after applying the transdermal delivery device, 72 hours after applying the transdermal delivery device, 76 hours after applying the transdermal delivery device, 80 hours after applying the transdermal delivery device, 84 hours after applying the transdermal delivery device, 96 hours after applying the transdermal delivery device, 120 hours after applying the transdermal delivery device and including 168 hours after applying the transdermal delivery device.

The transdermal flux of the tropane active agent may be determined one or more times at any given measurement period, such as 2 or more times, such as 3 or more times, including 5 or more times at each measurement period. An upper limit for the number of times the transdermal flux is determined is, in some instances, 10 times or fewer, such as 7 times or fewer, such as 5 times or fewer, such as 3 times or fewer and including 2 times or fewer. In certain embodiments, the number of times the transdermal flux of the tropane active agent is determined ranges such as from 2 times to 10 times, such as from 3 times to 9 times, such as from 4 times to 8 times and including from 5 times to 7 times.

In some embodiments, in maintaining the transdermal delivery device in contact with the subject the average cumulative amount of tropane active agent that permeates the skin of the subject increases at a substantially linear rate over the course of the dosage interval (e.g., 7 days or longer). By “substantially linearly” is meant that the cumulative amount of the tropane active agent released from the transdermal composition increases at a substantially constant rate (i.e., defined by zero-order kinetics). As such, the change in rate of cumulative tropane active agent permeated increases or decreases by 10% or less at any given time while maintaining the transdermal composition in contact with the subject, such as 8% or less, such as 7% or less, such as 6% or less, such as 5% or less, such as 3% or less, such as 2.5% or less, such as 2% or less, and including 1% or less at any time while maintaining the transdermal delivery device in contact with the subject.

In some embodiments, methods include maintaining the transdermal delivery devices in contact with the skin surface of the subject in a manner sufficient to deliver the tropane active agent to the subject:

at a substantially second order rate for a first predetermined period of time; and

at a substantially first order rate for a second predetermined period of time.

In these embodiments, the first period may be a duration ranging from 0.5 hours to 72 hours from administering the transdermal delivery device to the subject, such as from 1 hour to 60 hours, such as from 2 hours to 48 hours, such as from 3 hours to 36 hours, such as from 4 hours to 30 hours and including from 5 hours to 24 hours from the time of administration. The second period may be a duration ranging from 0.5 hours to 336 hours from administering the transdermal delivery device to the subject, such as from 1 hour to 312 hours, such as from 2 hours to 288 hours, such as from 3 hours to 264 hours, such as from 4 hours to 240 hours, such as from 5 hours to 216 hours and including from 6 hours to 192 hours from the time of administration. In certain instances, the first period of time is 24 hours or less, such as 20 hours or less, such as 16 hours or less, such as 12 hours or less, such as 8 hours or less and including 4 hours or less. In some instances, the second period of time is 1 hour or more, such as 2 hours or more, such as 4 hours or more, such as 8 hours or more, such as 12 hours or more, such as 16 hours or more, such as 20 hours or more and including 24 hours or more.

In other embodiments, methods include maintaining the transdermal delivery devices in contact with the skin surface of the subject in a manner sufficient to deliver the tropane active agent to the subject:

or pharmaceutically acceptable salt thereof to the subject:

at a substantially first order rate for a first predetermined period of time; and

at a substantially zero order rate for a second predetermined period of time.

In these embodiments, the first period may be a duration ranging from 0.5 hours to 72 hours from administering the transdermal delivery device to the subject, such as from 1 hour to 60 hours, such as from 2 hours to 48 hours, such as from 3 hours to 36 hours, such as from 4 hours to 30 hours and including from 5 hours to 24 hours from the time of administration. The second period may be a duration ranging from 0.5 hours to 336 hours from administering the transdermal delivery device to the subject, such as from 1 hour to 312 hours, such as from 2 hours to 288 hours, such as from 3 hours to 264 hours, such as from 4 hours to 240 hours, such as from 5 hours to 216 hours and including from 6 hours to 192 hours from the time of administration. In certain instances, the first period of time is 24 hours or less, such as 20 hours or less, such as 16 hours or less, such as 12 hours or less, such as 8 hours or less and including 4 hours or less. In some instances, the second period of time is 1 hour or more, such as 2 hours or more, such as 4 hours or more, such as 8 hours or more, such as 12 hours or more, such as 16 hours or more, such as 20 hours or more and including 24 hours or more.

In certain embodiments, each of the subject methods may further include the step of removing the transdermal delivery device from contact with the subject at the conclusion of a dosage interval. For example, the transdermal delivery device may be removed from contact with the subject after maintaining the transdermal delivery device in contact with the subject for 0.5 hours or more, such as 1 hour or more, such as 2 hours or more, such as 4 hours or more, such as 8 hours or more, such as 12 hours or more, such as 24 hours or more, such as 36 hours or more, such as 48 hours or more, such as 60 hours or more, such as 72 hours or more, such as 96 hours or more, such as 120 hours or more, including 144 hours or more, and including 168 hours or more. An upper limit for the amount of time the transdermal delivery device is maintained in contact with a subject before removal is, in some instances, 168 hours or shorter, such as 144 hours or shorter, such as 120 hours or shorter, such as 96 hours or shorter, such as 72 hours or shorter, such as 48 hours or shorter, such as 24 hours or shorter, such as 12 hours or shorter, such as 8 hours or shorter, such as 4 hours or shorter and including 2 hours or shorter.

By “removing” the transdermal delivery device from contact with the subject is meant that no amount of the tropane active agent from the transdermal composition remains in contact with the subject, including any residual amount of the tropane active agent left behind on the surface of the skin when the transdermal delivery device was applied. In other words, when the transdermal delivery device is removed all traces of the tropane active agent are no longer on the surface of the skin at the application site, resulting in zero transdermal flux of the tropane active agent into the subject.

As described above, a dosage interval is a single administration of applying and maintaining the transdermal delivery device in contact with the subject which begins with applying the transdermal delivery device to the skin of the subject and ends with the removal of the transdermal delivery device from contact with the subject. In certain embodiments, protocols for may include multiple dosage intervals. By “multiple dosage intervals” is meant more than one transdermal delivery device is applied and maintained in contact with the subject in a sequential manner. As such, a transdermal delivery device is removed from contact with the subject and a new transdermal delivery device is reapplied to the subject. In practicing methods of the invention, treatment regimens may include two or more dosage intervals, such as three or more dosage intervals, such as four or more dosage intervals, such as five or more dosage intervals, including ten or more dosage intervals.

The location on the subject for reapplying subsequent transdermal delivery devices in multiple dosage treatment regimens may be the same or different from the location on the subject where the previous transdermal delivery device was removed. For example, if a first transdermal delivery device is applied and maintained on the leg of the subject, one or more subsequent transdermal delivery devices may be reapplied to the same position on the leg of the subject. On the other hand, if a first transdermal delivery device was applied and maintained on the leg of the subject, one or more subsequent transdermal delivery device may be reapplied to a different position, such as the abdomen or back of the subject. Subsequent dosages applied in multiple dosage interval regimens may have the same or different formulation of the tropane active agent. In certain instances, a subsequent dosage interval in a treatment regimen may contain a higher or lower concentration of the tropane active agent than the previous dosage interval. For example, the concentration of the tropane active agent may be increased in subsequent dosage intervals by 10% or greater, such as 20% or greater, such as 50% or greater, such as 75% or greater, such as 90% or greater and including 100% or greater. An upper limit for the increase in concentration of the tropane active agent in subsequent dosage intervals is, in some instances, 10-fold or less, such as 5-fold or less, such as 2-fold or less, such as 1-fold or less, such as 0.5-fold or less and including 0.25-fold or less.

On the other hand, the concentration of the tropane active agent may be decreased in subsequent dosage intervals, such as by 10% or greater, such as 20% or greater, such as 50% or greater, such as 75% or greater, such as 90% or greater and including 100% or greater. An upper limit for the decrease in concentration of the tropane active agent in subsequent dosage intervals is, in some instances, 10-fold or less, such as 5-fold or less, such as 2-fold or less, such as 1-fold or less, such as 0.5-fold or less and including 0.25-fold or less.

In other instances, a subsequent dosage interval may contain a different formulation of the tropane active agent than the previous dosage interval, such as a different pressure sensitive adhesive or the presence or absence of a permeation enhancer, as described above.

In certain embodiments, compositions of the present disclosure can be administered prior to, concurrent with, or subsequent to other therapeutic agents for treating the same or an unrelated condition. If provided at the same time as another therapeutic agent, the subject transdermal compositions may be administered in the same or in a different composition. Thus, transdermal compositions having the tropane active agent and other therapeutic agents can be administered to the subject by way of concurrent therapy. By “concurrent therapy” is intended administration to a subject such that the therapeutic effect of the combination of the substances is caused in the subject undergoing therapy. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

Where the tropane active agent is administered concurrently with a second therapeutic agent to treat the same condition, the weight ratio of the tropane active agent to second therapeutic agent may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; and 1:10 and 1:25 or a range thereof. For example, the weight ratio of the tropane active agent to second therapeutic agent may range between 1:1 and 1:5; 1:5 and 1:10; 1:10 and 1:15; or 1:15 and 1:25. Alternatively, the weight ratio of the second therapeutic agent to the tropane active agent ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; and 10:1 and 25:1 ora range thereof. For example, the ratio of the second therapeutic agent the tropane active agent may range between 1:1 and 5:1; 5:1 and 10:1; 10:1 and 15:1; or 15:1 and 25:1.

### Utility

The subject transdermal delivery devices and methods for administering the tropane active agents find use in any application where a subject would benefit from inhibition of one or more of serotonin, norepinephrine or dopamine reuptake activity. In certain embodiments, transdermal delivery devices having an amount of a tropane active agent as described above find use in the treatment of cocaine addiction, obesity, diabetes, and obesity related disorders. Treating cocaine addiction includes at least a suppression or amelioration of the symptoms associated with the subject's dependence (e.g., physiological or psychological) on cocaine, where suppression and amelioration are used in a broad sense to refer to at least a reduction in the magnitude of a parameter, e.g., symptom, associated with the cocaine addiction. In some instances, the subject transdermal delivery devices find use in reducing or eliminating the physiological dependence on cocaine. In other instances, the subject transdermal delivery devices find use in reducing or eliminating the psychological dependence on cocaine. In yet other instances, the subject transdermal delivery devices find use in reducing or eliminating the physiological and psychological dependence on cocaine.

### Kits

Kits for use in practicing certain methods described herein are also provided. In certain embodiments, the kits include one or more extended transdermal delivery devices containing a therapeutically effective amount of a tropane active agent having one or more of serotonin, norepinephrine or dopamine reuptake inhibitor activity or pharmaceutically acceptable salt thereof and pressure sensitive adhesive as described above. In certain embodiments, the kits include an adhesive overlay as described above. In a given kit that includes two or more of the subject extended transdermal delivery devices, the compositions may be individually packaged or present within a common container.

In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable.

The following examples are offered by way of illustration and not by way of limitation. Specifically, the following examples are of specific embodiments for carrying out the present invention. The examples are for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### EXPERIMENTAL

### I. Materials and Methods

### A. Preparation of Example Transdermal Formulations of NS2359

Formulations were prepared by mixing stock solutions of each of the mixture components in organic solvents (typically 25-60 wt % solid content in ethyl acetate, isopropyl alcohol, hexane, or heptane), followed by mixing. Once a homogeneous mixture was formed, the solution was cast on a release liner (siliconized polyester sheet of 2-3 mils) and dried at 65°-80° C. for 10-90 minutes. The single layer adhesive films were then laminated to a PET backing, cut to the desired size, and pouched.

### B. Transdermal Flux Tests

Human cadaver skin was used and epidermal layers (stratum corneum and epidermis) were separated from the full-thickness skin as skin membrane. Samples were die-cut with an arch punch to a final diameter of about 2.0 cm². The release liner was removed and the system was placed on top of the epidermis/stratum corneum with the drug adhesive layer facing the stratum corneum. Gentle pressure was applied to effect good contact between the adhesive layer and stratum corneum. The donor and receptor sides of the Franz cell were clamped together and the receptor solution containing a phosphate buffer at pH 6.5 was added to the Franz cell. The cells were kept at 33° C. for the duration of the experiment. Samples of the receptor solution were taken at regular intervals and the active agent concentration was measured by HPLC. The removed receptor solution was replaced with fresh solution to maintain the sink conditions. The flux was calculated from the slope of cumulative amounts of the drug in the receiver compartment versus time plot.

### II. Results

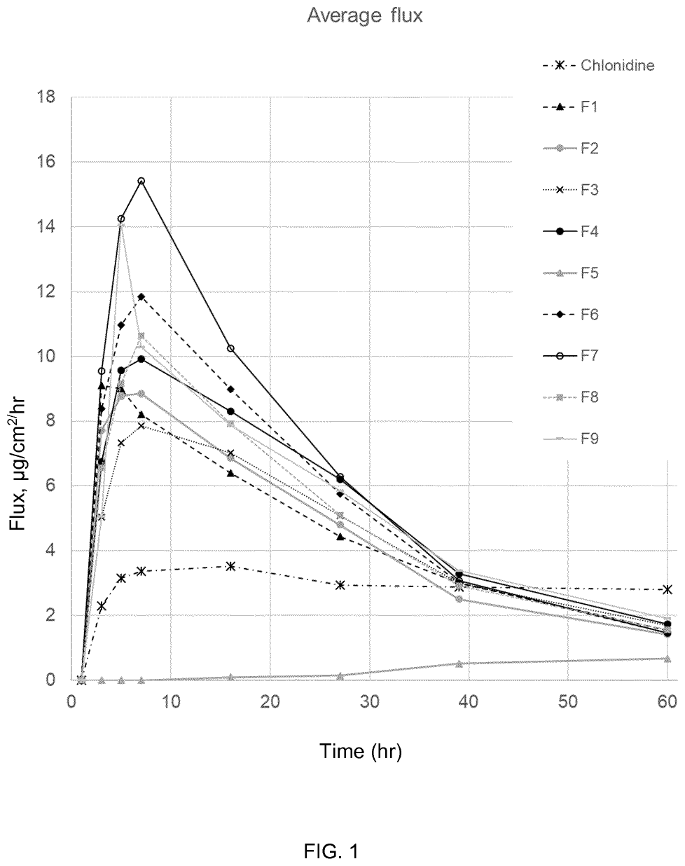

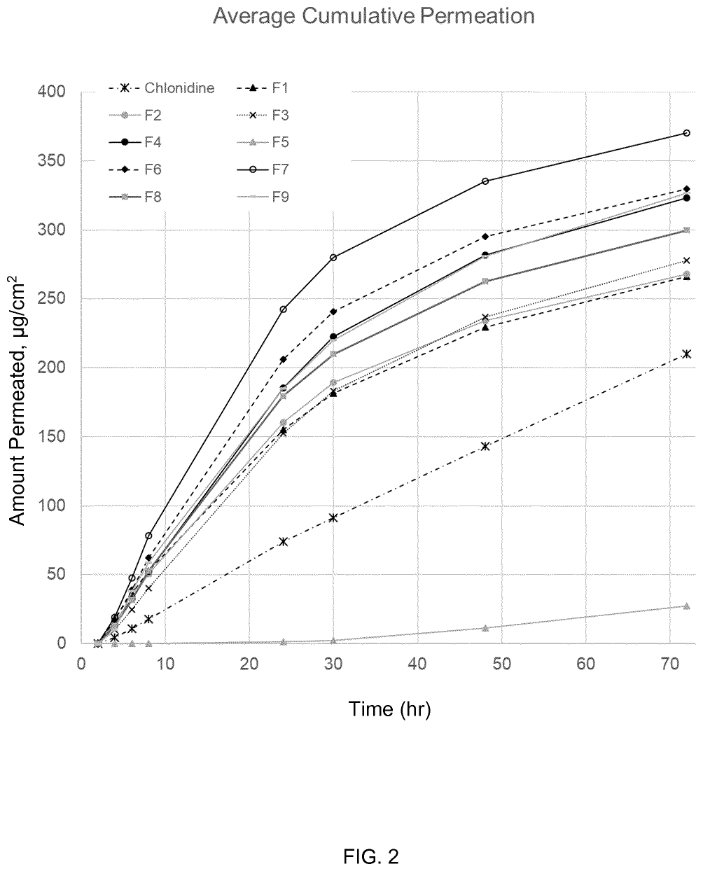

Transdermal delivery devices containing various formulations of (1 R,2 R,3 S,5 S)-3-(3,4-dichlorophenyl)-2-(methoxymethyl)-8-azabicyclo[3.2.1]octane (i.e., NS2359) are summarized in Table 1. An in vitro skin flux study was performed as described above with transdermal delivery devices having the formulations listed in Table 1. The transdermal delivery devices were compared with a reference patch containing transdermal clonidine. The flux rate of tropane compound (1 R,2 R,3 S,5 S)-3-(3,4-dichlorophenyl)-2-(methoxymethyl)-8-azabicyclo[3.2.1]octane from each of the formulations in Table 1 with respect to time is illustrated in FIG. 1, and the data is given in Table 2. As depicted in FIG. 1, the transdermal flux of (1 R,2 R,3 S,5 S)-3-(3,4-dichlorophenyl)-2-(methoxymethyl)-8-azabicyclo[3.2.1]octane reaches peak flux at about 8 hours for each of the formulations. Each of the formulations apart from that containing 10% w/w levulinic acid exhibit significantly greater transdermal flux as compared to clonidine. FIG. 2 depicts the average cumulative permeated amount of (1 R,2 R,3 S,5 S)-3-(3,4-dichlorophenyl)-2-(methoxymethyl)-8-azabicyclo[3.2.1]octane from each of formulations in Table 1 over time as compared to the clonidine patch (the data is given in Table 3). As depicted in FIG. 2, the tropane compound (1 R,2 R,3 S,5 S)-3-(3,4-dichlorophenyl)-2-(methoxymethyl)-8-azabicyclo[3.2.1]octane exhibits far greater average cumulative permeation from the formulations in Table 1 apart from that containing levulinic acid as compared to the clonidine patch. The results demonstrate that (1 R,2 R,3 S,5 S)-3-(3,4-dichlorophenyl)-2-(methoxymethyl)-8-azabicyclo[3.2.1]octane free base can be delivered transdermally.

TABLE 1
Composition of Formulations F1 to F9.

| Formulation No. | F1 | F2 | F3 | F4 | F5 | F6 | F7 | F8 | F9 |
|---|---|---|---|---|---|---|---|---|---|
| (1R,2R,3S,5S)-3-(3,4- | 5 | 5 | 5 | 5 | 5 | 5 | 5 | 5 | 5 |
| dichloropheny1)-2- | | | | | | | | | |
| (methoxymethyl)-8- | | | | | | | | | |
| azabicyclo[3.2.1]octane | | | | | | | | | |
| (% wt/wt) | | | | | | | | | |
| PVP (% wt/wt) | — | 5 | 10 | 10 | 10 | 10 | 10 | 10 | — |
| Lauryl Lactate (% wt/wt) | — | — | — | 8 | — | — | — | — | — |
| Levulinic Acid (% wt/wt) | — | — | — | — | 10 | — | — | — | — |
| Glyceryl monooleate | — | — | — | — | — | 8 | — | — | — |
| (GMO) (% wt/wt) | | | | | | | | | |
| Dodecyl pyrrolidone | — | — | — | — | — | — | 10 | — | — |
| (DDP) (% wt/wt) | | | | | | | | | |
| Hexylene Glycol | — | — | — | — | — | — | — | 10 | — |
| (HexGly) (% wt/wt) | | | | | | | | | |
| Duro-tak 87-9301 | 95 | 90 | 85 | 77 | 75 | 77 | 75 | 75 | — |
| (% wt/wt) | | | | | | | | | |
| Duro-tak 87-900A | — | — | — | — | — | — | — | — | 95 |
| (% wt/wt) | | | | | | | | | |

TABLE 2
Flux rate of tropane compound (1R,2R,3S,5S)-3-(3,4-dichloropheny1)-2-
(methoxymethyl)-8-azabicyclo[3.2.1]octane from each of the
formulations in Table 1 with respect to time (μg/cm²/hr).

| Time | Formulation | | | | | | | | | |
|---|---|---|---|---|---|---|---|---|---|---|
| (hr) | Chlonidine | F1 | F2 | F3 | F4 | F5 | F6 | F7 | F8 | F9 |
| 1 | 0 | 0.04 | 0.01 | 0 | 0 | 0 | 0.02 | 0 | 0 | 0 |
| 3 | 2.29 | 9.1 | 7.71 | 5.03 | 6.75 | 0 | 8.38 | 9.54 | 6.56 | 5.06 |
| 5 | 3.15 | 9.01 | 8.77 | 7.32 | 9.57 | 0 | 10.96 | 14.25 | 9.15 | 14.06 |
| 7 | 3.36 | 8.21 | 8.85 | 7.86 | 9.91 | 0 | 11.83 | 15.41 | 10.64 | 10.29 |
| 16 | 3.51 | 6.39 | 6.86 | 7.02 | 8.31 | 0.09 | 8.99 | 10.25 | 7.92 | 7.88 |
| 27 | 2.94 | 4.43 | 4.8 | 5.07 | 6.2 | 0.14 | 5.75 | 6.27 | 5.09 | 5.85 |
| 39 | 2.88 | 2.99 | 2.5 | 2.99 | 3.28 | 0.51 | 3.01 | 3.08 | 2.92 | 3.38 |
| 60 | 2.79 | 1.54 | 1.41 | 1.7 | 1.74 | 0.67 | 1.45 | 1.45 | 1.55 | 1.91 |

TABLE 3
Average cumulative permeated amount of (1R,2R,3S,5S)-3-(3,4-
dichloropheny1)-2-(methoxymethyl)-8-azabicyclo[3.2.1]octane from each
of formulations in Table 1 over time as compared to the clonidine patch (μg/cm²).

| Time | Formulation | | | | | | | | | |
|---|---|---|---|---|---|---|---|---|---|---|
| (hr) | Chlonidine | F1 | F2 | F3 | F4 | F5 | F6 | F7 | F8 | F9 |
| 2 | 0.01 | 0.09 | 0.02 | 0 | 0 | 0 | 0.04 | 0 | 0 | 0 |
| 4 | 4.59 | 18.29 | 15.43 | 10.07 | 13.5 | 0 | 16.81 | 19.08 | 13.12 | 10.11 |
| 6 | 10.89 | 36.3 | 32.96 | 24.71 | 32.65 | 0 | 38.74 | 47.58 | 31.43 | 38.23 |
| 8 | 17.62 | 52.72 | 50.65 | 40.43 | 52.48 | 0 | 62.4 | 78.4 | 52.72 | 58.81 |
| 24 | 73.73 | 155.03 | 160.37 | 152.75 | 185.51 | 1.49 | 206.28 | 242.43 | 179.41 | 184.84 |
| 30 | 91.37 | 181.61 | 189.16 | 183.16 | 222.72 | 2.34 | 240.76 | 280.04 | 209.95 | 219.91 |
| 48 | 143.16 | 229.29 | 234.24 | 236.97 | 281.72 | 11.43 | 294.92 | 335.46 | 262.59 | 280.84 |
| 72 | 210.04 | 266.13 | 268.03 | 277.71 | 323.39 | 27.41 | 329.72 | 370.14 | 299.7 | 326.72 |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A transdermal delivery device configured to transdermally deliver to a subject a therapeutically effective amount of a tropane active agent of Formula I: 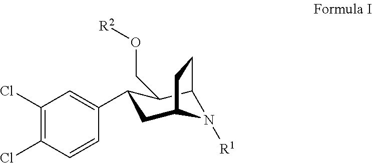
where
R¹=—H or —CH₃, and
R²=—CH₃ or —CH₂CH₃
or a pharmaceutically acceptable salt thereof.

2. The transdermal delivery device according to claim 1, wherein the tropane active agent is (1 R,2 R,3 S,5 S)-3-(3,4-dichlorophenyl)-2-(methoxymethyl)-8-azabicyclo[3.2.1]octane or a pharmaceutically acceptable salt thereof.

3. The transdermal delivery device according to claim 1, wherein the tropane active agent is (1 R,2 R,3 S,5 S)-3-(3,4-dichlorophenyl)-2-(ethoxymethyl)-8-methyl-8-azabicyclo[3.2.1]octane or a pharmaceutically acceptable salt thereof.

4. The transdermal delivery device according to claim 1, wherein the transdermal delivery device comprises:
an active agent layer comprising:
the tropane active agent; and
a pressure sensitive adhesive; and
a backing layer.

5-8. (canceled)

9. The transdermal delivery device according to claim 4, wherein the pressure sensitive adhesive of the active agent layer is an acrylate copolymer.

10-11. (canceled)

12. The transdermal delivery device according to claim 4, wherein the active agent layer further comprises a hydrophilic polymer comprising polyvinylpyrrolidone or crosslinked polyacrylic acid.

13-17. (canceled)

18. The transdermal delivery device according to claim 4, wherein the active agent layer further comprises one or more compounds selected from the group consisting of polyvinylpyrrolidone, lauryl lactate, glyceryl monooleate, dodecyl pyrrolidone, and hexylene glycol.

19. (canceled)

20. The transdermal delivery device according to claim 4, wherein the active agent layer comprises 2 to 10% wt/wt tropane active agent, 2 to 15% wt/wt hydrophilic polymer comprising polyvinylpyrrolidone, and 60 to 80% wt/wt acrylate copolymer.

12-37. (canceled)

38. A method of transdermal delivery of a tropane active agent of Formula I: 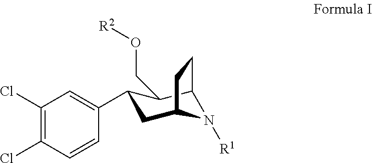
where
R¹=—H or —CH_{3,} and
R²=—CH₃ or —CH₂CH₃ or a pharmaceutically acceptable salt thereof to a subject, said method comprising applying a transdermal delivery device comprising the tropane active agent to a skin surface of the subject in a manner sufficient to deliver a therapeutically effective amount of the tropane active agent to the subject.

39. The method according to claim 38, wherein the tropane active agent is (1 R,2 R,3 S,5 S)-3-(3 ,4-dichlorophenyl)-2-(methoxymethyl)-8-azabicyclo[3 .2.1]octane or a pharmaceutically acceptable salt thereof.

40. The method according to claim 38, wherein the tropane active agent is (1 R,2 R,3 S,5 S)-3-(3 ,4-dichlorophenyl)-2-(ethoxymethyl)-8-methyl-8-azabicyclo[3 .2.1]octane or a pharmaceutically acceptable salt thereof.

41. The method according to claim 38, wherein the method comprises maintaining the transdermal delivery device in contact with the skin surface for 1 day or less.

42. (canceled)

43. The method according to claim 38, wherein the method comprises maintaining the transdermal delivery device in contact with the skin surface for 3 days or more.

44-46. (canceled)

47. The method according to claim 38, wherein the transdermal delivery device comprises:
an active agent layer comprising:
the tropane active agent; and
a pressure sensitive adhesive; and
a backing layer.

48-51. (canceled)

52. The method according to claim 47, wherein the pressure sensitive adhesive of the active agent layer is an acrylate copolymer.

53-54. (canceled)

55. The method according to claim 47, wherein the active agent layer further comprises a hydrophilic polymer comprising polyvinylpyrrolidone or crosslinked polyacrylic acid.

56-60. (canceled)

61. The method according to claim 47, wherein the active agent layer further comprises one or more compounds selected from the group consisting of polyvinylpyrrolidone, lauryl lactate, glyceryl monooleate, dodecyl pyrrolidone, and hexylene glycol.

62. (canceled)

63. The method according to claim 47, wherein the active agent layer comprises 2 to 10% wt/wt tropane active agent, 2 to 15% wt/wt hydrophilic polymer comprising polyvinylpyrrolidone, and 60 to 80% wt/wt acrylate copolymer.

64-79. (canceled)

80. A method of treating a subject for cocaine addiction, said method comprising applying to a skin surface of the subject the transdermal device according to claim 1, wherein the tropane active agent is (1 R,2 R,3 S,5 S)-3-(3,4-dichlorophenyl)-2-(methoxymethyl)-8-azabicyclo[3.2.1]octane or a pharmaceutically acceptable salt thereof.

81-83. (canceled)

84. A method for treatment of obesity or an obesity related disorder of a subject, said method comprising applying to a skin surface of the subject the transdermal device according to claim 1, wherein the tropane active agent is (1 R,2 R,3 S,5 S)-3-(3 ,4-dichlorophenyl)-2-(ethoxymethyl)-8-methyl-8-azabicyclo[3 .2.1]octane or a pharmaceutically acceptable salt thereof.

85-88. (canceled)
